(19) United States
(12) Patent Application Publication
(10) Pub. No.: US 2026/0007554 A1
(43) Pub. Date: Jan. 8, 2026

(54) PATIENT SUPPORT APPARATUS SYSTEM

(71) Applicant: Stryker Corporation, Portage, MI (US)

(72) Inventors: Michael W. Graves, Augusta, MI (US); Matthew A. Cutler, Mattawan, MI (US); Christopher Ryan Sweeney, Portage, MI (US); Lavanya Vytla, Prosper, TX (US); Krishna Sandeep Bhimavarapu, Kalamazoo, MI (US); Brianna R. Graves, Paw Paw, MI (US); Jeffrey Kennedy, Kalamazoo, MI (US); Matthew E. Harrow, Mooresville, NC (US)

(21) Appl. No.: 19/149,798

(22) PCT Filed: Feb. 8, 2024
(86) PCT No.: PCT/US2024/014966

§ 371 Date: Jul. 21, 2025

Related U.S. Application Data
(60) Provisional application No. 63/444,415, filed on Feb. 9, 2023.
(60) Provisional application No. 63/615,442, filed on Dec. 28, 2023.

(51) Int. Cl. A61G 7/05; A61G 7/012; G10L 15/08; G10L 15/22; G10L 15/30; G16H 40/67

(52) U.S. Cl. CPC A61G 7/0526; G10L 15/08; G10L 15/22; G10L 15/30; G16H 40/67; A61G 7/012; A61G 7/0527; A61G 7/0528; A61G 2203/10; A61G 2203/16; A61G 2203/18; A61G 2203/30; G10L 2015/088

(57) ABSTRACT

A patient support apparatus includes a support surface, a control panel, a network transceiver, and a controller. The controller is adapted to send one or more messages to a server that indicates at least one of the following: an incorrect passcode was entered into the control panel; a correct passcode was entered into the control panel; a post-passcode-entry window of time expired; a microphone detected a sound level exceeding a threshold; the microphone detected a keyword; a block control was activated on the patient support apparatus; an agitation level of the patient; a patient activation of a nurse call control; a status of a patient restraint; a status of a restraint attachment cover; or movement of the patient support apparatus as detected by an accelerometer. A software application executed by a server may be configured to instruct a display to display an indicator relating to any of these messages.

### BACKGROUND

The present disclosure relates to patient support apparatuses, such as beds, cots, stretchers, recliners, or the like. More specifically, the present disclosure relates to a system for sharing information regarding patient support apparatuses with caregivers who are positioned remotely from the patient support apparatuses.

Hospitals typically expect nurses and/or other caregivers to perform a variety of different duties when caring for patients. In some situations, these duties include, in addition to caring for the patient, monitoring the patient's behavior in order to help prevent the patient from harming themselves or other, and/or causing damage to property.

### SUMMARY

According to various embodiments, a system is provided that assists caregivers with the task of monitoring patients that may be at risk of self-harm or at risk of harming property or other personnel within a healthcare setting. The system includes a patient support apparatus that may include, or be in communication with, one or more sensors that detect one or more of the following conditions: an incorrect passcode was entered into a control panel on the patient support apparatus; a correct passcode was entered into the control panel; the window of time expired; a microphone on the patient support apparatus detected a sound level exceeding a threshold; a microphone on the patient support apparatus detected a keyword; a block control was activated on the patient support apparatus wherein the block control prevents access to the plurality of functions until the correct passcode is entered; an agitation level of a patient onboard the patient support apparatus; a patient activation of a nurse call control onboard the patient support apparatus; a restraint status indicative of whether the patient is currently restrained or not; a restraint cover status indicative of whether a restraint attachment on the patient support apparatus is currently covered or not; or movement of the patient support apparatus as detected by an accelerometer. In response to detecting one or more of these and/or other conditions, the patient support apparatus may send a message to a remote server that is adapted to instruct a display device to display one or more indicators indicative of these conditions. The display device may be a smart phone carried by caregivers, a caregiver badge, a display at a nursing station or other location, or another type of display. Remotely positioned caregivers are thereby alerted of conditions that may indicate an increased risk of a patient causing harm to themselves, to others, and/or to property. A hostile person detection system that may also and/or alternatively implemented in a healthcare environment is also disclosed. The hostile person detection system automatically analyzes peoples voices, determines a sentiment of the persons, and issues an alert to appropriate personnel if a person is potentially hostile. These and other features and functions of the system will be apparent to one skilled in the art in light of the following written description and the attached drawings.

According to a first embodiment of the present disclosure, a patient support apparatus is provided that includes a support surface, a control panel, a network transceiver, and a controller. The support surface is adapted to support a patient thereon. The control panel is adapted to allow access to a plurality of functions of the patient support apparatus for a window of time after a correct passcode is entered into the control panel, and to disallow access to the plurality of functions after the window of time expires. The network transceiver is adapted to communicate with a healthcare facility computer network. The controller is adapted to send a first message to the healthcare facility computer network when the correct passcode is entered into the control panel and to send a second message to the healthcare facility computer network when the window of time expires.

According to another aspect of the present disclosure, the controller is further adapted to send a third message to the healthcare facility computer network when an incorrect passcode is entered into the control panel. The third message indicates that an incorrect passcode was entered into the control panel.

In some aspects, the control panel includes a touchscreen and the control panel is adapted to display a passcode entry screen on the touchscreen.

The patient support apparatus, in some aspects, includes a microphone and the controller is adapted to send a third message to the healthcare facility computer network when a sound level detected by the microphone exceeds a threshold.

The controller, in some aspects, is adapted to allow a user to select a value for the threshold.

The controller, in some aspects, is adapted to allow a user to select different values for the threshold for different times of a day.

The controller, in some aspects, is controller is further adapted to perform speech recognition on sounds detected by the microphone and to send a third message to the healthcare facility computer network if the controller recognizes speech of a keyword.

The controller, in some aspects, may further be adapted to send the third message to the healthcare facility computer network if the controller recognizes speech of any one or more of a plurality of keywords.

In some aspects, the controller is adapted to allow a user to change the keyword.

The window of time, in some aspects, is defined as a set time period during which the control panel is not used.

In some aspects, the patient support apparatus further includes a plurality of additional control panels and the window of time is defined as a set time period during which neither the control panel nor any of the plurality of additional control panels are used.

In some aspects, the control panel includes a block control and, in response to a user activating the block control, the window of time expires and the control panel is adapted to block access to the plurality of functions.

The first message, in some aspects, indicates that the correct passcode was entered and the second message indicates that the control panel has disallowed access to the plurality of functions.

The plurality of functions, in some aspects, include a plurality of motion lockouts, and each of the motion lockouts is adapted to disable particular movement of the patient support apparatus when activated and to enable the particular movement of the patient support apparatus when deactivated.

The patient support apparatus, in some aspects, includes a caregiver presence sensor adapted to detect a presence of a caregiver within a vicinity of the patient support apparatus. The controller is further adapted to send a third message to the healthcare facility computer network when the caregiver presence sensor detects the presence of the caregiver within the vicinity of the patient support apparatus.

In some aspects, the patient support apparatus includes a caregiver presence sensor adapted to detect a presence of a caregiver within a vicinity of the patient support apparatus, and the controller is further adapted to send a third message to the healthcare facility computer network when the caregiver presence sensor detects an absence of the caregiver within the vicinity of the patient support apparatus.

In some aspects, the patient support apparatus includes a microphone and the controller is adapted to determine a filtered sound reading by filtering out a set of frequencies from sound detected by the microphone, to compare the filtered sound reading to a threshold, and to send a third message to the healthcare facility computer network if the filtered sound reading exceeds a threshold.

The patient support apparatus, in some aspects, includes a plurality of force sensors adapted to detect downward forces exerted on the support surface, and the controller is adapted to use outputs from the plurality of force sensors to determine both a patient weight and an agitation level of the patient.

In some aspects, the controller is further adapted to transmit the agitation level to the healthcare facility computer network.

In some aspects, the controller is adapted to also use audio signals generated by the microphone when determining the agitation level of the patient.

The patient support apparatus, in some aspects, further includes a camera having a field of view that captures at least a portion an area above the support surface, and the controller is adapted to also analyze images from the camera when determining the agitation level of the patient.

The patient support apparatus, in some aspects, further includes a nurse call control that is adapted to call a remotely positioned nurse, and the controller is adapted to keep track of a number of times the patient uses the nurse call control and to use that number when determining the agitation level of the patient.

In some aspects, the controller is adapted to keep track of a number of times the patient enters an incorrect passcode and to use that number when determining the agitation level of the patient.

The controller, in some aspects, is adapted to keep track of a number of times the patient activates a motion control on the patient support apparatus, and to use that number when determining the agitation level.

In some aspects, the patient support apparatus further includes a CPR control adapted to automatically bring the support surface to a flat orientation when activated, and the controller is adapted to use activation of the CPR control when determining the agitation level.

According to another embodiment of the present disclosure, a software application is provided that is embodied in a non-transitory computer readable medium and that is adapted to, when executed by a processor of a computing device, to cause the computing device to perform the following: receive a first message from a patient support apparatus wherein the first message indicates that the patient support apparatus has granted user access to a plurality of functions of the patient support apparatus; receive a second message from the patient support apparatus wherein the second message indicates that the patient support apparatus has denied user access to the plurality of functions of the patient support apparatus; and instruct a display device to display a first indicator in response to receiving the first message. The first indicator indicates that user access has been granted to the plurality of functions of the patient support apparatus.

According to other aspects of the present disclosure, the software application is further adapted to instruct the display device to display a second indicator in response to receiving the second message, wherein the second indicator indicates that user access has been denied to the plurality of functions.

In some aspects, the software application is further adapted to instruct the computing device to receive the first message in response to a user entering a correct passcode into a control panel of the patient support apparatus.

The software application, in some aspects, is further adapted to instruct the computing device to receive the second message in response to a window of time expiring.

In some aspects, the software application is further adapted to instruct the computing device to receive a third message from the patient support apparatus indicating that a caregiver is currently present within a vicinity of the patient support apparatus. The software application is further adapted to instruct the display device to display a third indicator in response to receiving both the first message and the third message.

The software application, in some aspects, is further adapted to instruct the computing device to receive a third message indicating an incorrect passcode was entered into the control panel, and to instruct a display to display a third indicator indicating that an incorrect passcode was entered into the control panel.

In some aspects, the software application is further adapted to instruct the computing device to receive a third message indicating a microphone on the patient support apparatus detected a sound level in excess of a threshold, and to instruct a display to display a third indicator indicating that the sound level exceeded the threshold.

The software application, in some aspects, is further adapted to instruct the computing device to receive a third message indicating the patient support apparatus has recognized a patient speaking a keyword, and to instruct a display to display a third indicator indicating that the keyword has been spoken.

In some aspects, the software application is further adapted to instruct the computing device to receive a third message indicating the patient support apparatus has recognized a patient speaking any one of a plurality of keywords, and to instruct a display to display a third indicator indicating that one or more of the plurality of keywords has been spoken.

The software application, in some aspects, is further adapted to instruct the computing device to receive a third message from the patient support apparatus indicating that a caregiver is currently present within a vicinity of the patient support apparatus, and to also instruct the display device to not display the first indicator in response to receiving the third message.

In some aspects, the software application is further adapted to instruct the computing device to receive a third message from the patient support apparatus indicating an agitation level of a patient, and to instruct a display to display a third indicator indicating the agitation level.

In some aspects, the software application is further adapted to instruct the computing device to receive a third message from the patient support apparatus indicating an agitation level of a patient, and to instruct a display to display a third indicator if the agitation level is above a threshold.

The software application, in some aspects, is further adapted to instruct the computing device to receive a third message generated from analysis of an image captured by a camera, wherein the analysis of the image determines a restraint status of a patient and the restraint status indicates whether the patient is currently restrained on the patient support apparatus or not. The software application is further adapted to instruct the display to display a third indicator indicating the restraint status.

The software application, in some aspects, is further adapted to instruct the computing device to receive a third message generated from analysis of an image captured by a camera, wherein the analysis of the image determines a restraint cover status and the restraint cover status indicates whether a restraint attachment on the patient support apparatus is currently covered or not, and wherein the software application is further adapted to instruct the display to display a third indicator indicating the restraint cover status.

In some aspects, the software application is further adapted to instruct the computing device to determine an agitation level of the patient using at least two of the following: outputs from a microphone onboard the patient support apparatus; analysis of images having a field of view that captures at least a portion of an area above a support surface of the patient support apparatus; a number of times the patient uses a nurse call control on the patient support apparatus; a number of times a patient enters an incorrect passcode into the patient support apparatus; a number of times the patient activates a motion control on the patient support apparatus; outputs from a plurality of force sensors positioned onboard the patient support apparatus; a vital sign of the patient; a door alarm; or outputs from an accelerometer onboard the patient support apparatus.

The computing device, in some aspects, is a server hosted on a network of a healthcare facility.

According to another aspect of the present disclosure, a patient support apparatus is provided that includes a support surface, a network transceiver, and a controller. The support surface is adapted to support a patient thereon. The network transceiver is adapted to communicate with a healthcare facility computer network. The controller is adapted to send a first message to the healthcare facility computer network that indicates a first condition. The first condition includes at least one of the following: an incorrect passcode was entered into a control panel on the patient support apparatus; a correct passcode was entered into the control panel; a window of time since any control panel on the patient support apparatus was last used has expired; a microphone on the patient support apparatus detected a sound level exceeding a threshold; a microphone on the patient support apparatus detected a keyword; a block control was activated on the patient support apparatus wherein the block control prevents access to the plurality of functions until the correct passcode is entered; an agitation level of a patient onboard the patient support apparatus; a patient activation of a nurse call control onboard the patient support apparatus; a restraint status indicative of whether the patient is currently restrained or not; a restraint cover status indicative of whether a restraint attachment on the patient support apparatus is currently covered or not; or movement of the patient support apparatus as detected by an accelerometer.

According to other aspects of the present disclosure, the controller is further adapted to send a second message to the healthcare facility computer network that indicates a second condition and the second condition includes at least one other of the following: an incorrect passcode was entered into the control panel; a correct passcode was entered into the control panel; the window of time expired; a microphone on the patient support apparatus detected a sound level exceeding a threshold; a microphone on the patient support apparatus detected a keyword; a block control was activated on the patient support apparatus wherein the block control prevents access to the plurality of functions until the correct passcode is entered; an agitation level of a patient onboard the patient support apparatus; a patient activation of a nurse call control onboard the patient support apparatus; a restraint status indicative of whether the patient is currently restrained or not; a restraint cover status indicative of whether a restraint attachment on the patient support apparatus is currently covered or not; or movement of the patient support apparatus as detected by an accelerometer.

In some aspects, the patient support apparatus further includes a control panel adapted to allow access to a plurality of functions of the patient support apparatus for a window of time after a correct passcode is entered into the control panel, and to disallow access to the plurality of functions after the window of time expires. The control panel includes a touchscreen and the control panel is adapted to display a passcode entry screen on the touchscreen.

In some aspects, the patient support apparatus includes a microphone and the first message indicates a sound level exceeding a threshold was detected by the microphone.

In some aspects, the patient support apparatus includes a microphone and the first message indicates a keyword was detected by the microphone.

The controller, in some aspects, is adapted to allow a user to change the keyword.

The first message, in some aspects, indicates the window of time expired.

In some aspects, the first message indicates that the correct passcode was entered and the second message indicates that the window of time has expired.

The plurality of functions, in some aspects, includes a plurality of motion lockouts and each of the motion lockouts is adapted to disable particular movement of the patient support apparatus when activated and to enable the particular movement of the patient support apparatus when deactivated.

The patient support apparatus, in some aspects, includes a caregiver presence sensor adapted to detect a presence of a caregiver within a vicinity of the patient support apparatus. The controller is further adapted to send a third message to the healthcare facility computer network when the caregiver presence sensor detects the presence of the caregiver within the vicinity of the patient support apparatus.

The patient support apparatus, in some aspects, includes a caregiver presence sensor adapted to detect a presence of a caregiver within a vicinity of the patient support apparatus, and the controller is further adapted to send a third message to the healthcare facility computer network when the caregiver presence sensor detects an absence of the caregiver within the vicinity of the patient support apparatus.

In some aspects, the first message indicates an agitation level of the patient.

The patient support apparatus, in some aspects, includes a plurality of force sensors adapted to detect downward forces exerted on the support surface, and the controller is adapted to use outputs from the plurality of force sensors to determine both a patient weight and an agitation level of the patient.

In some aspects, the controller is adapted to use audio signals generated by the microphone when determining the agitation level of the patient.

In some aspects, the controller is adapted to communicate with a camera having a field of view that encompasses at least a portion an area above the support surface, and the controller is further adapted to also analyze images from the camera when determining the agitation level of the patient.

In some aspects, the camera is integrated into the patient support apparatus, supported on the patient support apparatus but not integrated therein, or positioned or off-board the patient support apparatus.

The patient support apparatus, in some aspects, includes a nurse call control that is adapted to call a remotely positioned nurse, and the controller is adapted to keep track of a number of time the patient uses the nurse call control and to use that number when determining the agitation level of the patient.

The controller, in some aspects, is adapted to keep track of a number of times the patient enters an incorrect passcode and to use that number when determining the agitation level of the patient.

The controller, in some aspects, is adapted to keep track of a number of times the patient activates a motion control on the patient support apparatus, and to use that number when determining the agitation level.

In some aspects, the patient support apparatus includes a CPR control adapted to automatically bring the support surface to a flat orientation when activated, and the controller is adapted to use activation of the CPR control when determining the agitation level.

According to another embodiment of the present disclosure, a software application is provided that is embodied in a non-transitory computer readable medium and that is adapted to, when executed by a processor of a computing device, to cause the computing device to perform the following: receive a first message from a patient support apparatus and instruct a display device to display a first indicator in response to receiving the first message. The first message indicates a first condition that includes at least one of the following: an incorrect passcode was entered into a control panel of the patient support apparatus; a correct passcode was entered into the control panel; a window of time since any control panel on the patient support apparatus was last used has expired; a microphone on the patient support apparatus detected a sound level exceeding a threshold; a microphone on the patient support apparatus detected a keyword; a block control was activated on the patient support apparatus wherein the block control blocks access to a plurality of functions of the patient support apparatus until the correct passcode is entered; an agitation level of a patient onboard the patient support apparatus; a patient activation of a nurse call control onboard the patient support apparatus; a restraint status indicative of whether the patient is currently restrained or not; a restraint cover status indicative of whether a restraint attachment on the patient support apparatus is currently covered or not; or movement of the patient support apparatus as detected by an accelerometer.

According to another aspect of the present disclosure, the software application is further adapted to instruct the computing device to receive a second message from the patient support apparatus, wherein the second message indicates a second condition and the second condition includes at least one other of the following: an incorrect passcode was entered into the control panel; the correct passcode was entered into the control panel; the window of time expired; the microphone on the patient support apparatus detected a sound level exceeding the threshold; the microphone on the patient support apparatus detected the keyword; the block control was activated on the patient support apparatus; the agitation level of the patient onboard the patient support apparatus; the patient activation of the nurse call control onboard the patient support apparatus; the restraint status indicative of whether the patient is currently restrained or not; the restraint cover status indicative of whether the restraint attachment on the patient support apparatus is currently covered or not; or movement of the patient support apparatus as detected by the accelerometer.

In some aspects, the software application is further adapted to instruct the display device to display a second indicator in response to receiving the second message.

The first message, in some aspects, indicates that the sound level detected by the microphone on the patient support apparatus exceeded the threshold.

The first message, in some aspects, indicates that the keyword was detected by the microphone on the patient support apparatus.

The first message, in some aspects, indicates that the window of time has expired.

The first message, in some aspects, indicates that the correct passcode was entered and the second message indicates that the window of time has expired.

The first message, in some aspects, indicates an agitation level of the patient.

The agitation level, in some aspects, is determined from force sensors on the patient support apparatus adapted to measure a patient weight.

In some aspects, the agitation level is determined from the microphone.

In some aspects, the agitation level is determined from an analysis of images captured by a camera having a field of view that encompasses at least a portion an area above a support surface of the patient support apparatus.

In some aspects, the agitation level is determined from a number of times the patient uses a nurse call control on the patient support apparatus to call a remotely positioned nurse.

In some aspects, the agitation level is determined from a number of times the patient enters an incorrect passcode.

In some aspects, the agitation level is determined from activation of a CPR control on the patient support apparatus by the patient.

In some aspects, the agitation level is determined from any two or more of the following: (a) force sensors on the patient support apparatus adapted to measure a patient weight; (b) the microphone; (c) an analysis of images captured by a camera having a field of view that encompasses at least a portion an area above a support surface of the patient support apparatus; (d) a number of time the patient uses a nurse call control on the patient support apparatus to call a remotely positioned nurse; (e) a number of times the patient enters an incorrect passcode; or (f) activation of a CPR control on the patient support apparatus by the patient.

According to another aspect of the present disclosure, a system is provided for automatically detecting a person's hostility in a healthcare facility. The system comprises a first microphone, a transmitter, and a computing device. The first microphone is adapted to output first audio signals representative of sounds detected by the first microphone, the sounds including background noise and the person's voice. The transmitter is adapted to transmit the first audio signals. The computing device is adapted to receive the transmitted first audio signals, separate the person's voice from the background noise, perform a sentiment analysis on the person's voice to determine an emotional state of the person, and to transmit a warning message to a healthcare facility worker warning the healthcare facility worker of a potential threat of hostility of the person if the emotional state is a negative state.

According to other aspects of the present disclosure, the computing device is adapted to convert the person's voice to words and to utilize the words when performing the sentiment analysis.

In some aspects, the first microphone is integrated into a badge adapted to be worn by a caregiver in the healthcare facility.

The transmitter, in some aspects, is a wireless transmitter adapted to transmit the first audio signals to a local area network of the healthcare facility.

In some aspects, the system includes a badge in communication with the computing device, wherein the badge includes a second microphone adapted to output second audio signals representative of sounds detected by the second microphone, and a second transmitter adapted to transmit the second audio signals.

The badge, in some aspects, includes a first ultra-wideband (UWB) transceiver adapted to range with a second UWB transceiver positioned at a known location, and the computing device is adapted to determine a location of the badge based on the ranging between the first and second UWB transceivers.

The system, in some aspects, includes a camera adapted to record video images of an area of the healthcare facility and to transmit the video images to the computing device. The computing device is adapted to superimpose an indicator on the video images at a location corresponding to the person.

In some aspects, the system includes a patient support apparatus in communication with the computing device. The patient support apparatus includes a second microphone adapted to output second audio signals representative of sounds detected by the second microphone, and a second transmitter adapted to transmit the second audio signals.

In some aspects, the patient support apparatus includes a first ultra-wideband (UWB) transceiver adapted to range with a second UWB transceiver positioned at a known location, and the computing device is adapted to determine a location of the patient support apparatus based on the ranging between the first and second UWB transceivers.

The system, in some aspects, includes a camera adapted to record video images of an area of the healthcare facility and to transmit the video images to the computing device, and the computing device is adapted to superimpose an indicator on the video images at a location corresponding to the person.

The transmitter, in some aspects, is adapted to transmit location information indicative of a location of the first microphone, and the computing device is adapted to identify a room in the healthcare facility in which the first microphone is positioned based on the location information.

The computing device, in some aspects, is adapted to use the room to select which healthcare facility worker to transmit the warning message to.

In some aspects, the computing device is further adapted to include the room in the warning message transmitted to the healthcare facility worker.

The system, in some aspects, includes a locator unit in communication with the computing device and adapted to transmit a location signal to a nearby device. The locator unit includes a second microphone adapted to output second audio signals representative of sounds detected by the second microphone, and a second transmitter adapted to transmit the second audio signals.

The computing device, in some aspects, is adapted to receive the second audio signals and triangulate a position of the person using the first and second audio signals.

In some aspects, the locator unit includes a camera adapted to record video images of an area of the healthcare facility and to transmit the video images to the computing device. The computing device is further adapted to superimpose an indicator on the video images at a location corresponding to the person.

The locator unit, in some aspects, is adapted to transmit location information indicative of a location of the locator unit, and the computing device is adapted to identify a room in the healthcare facility in which the locator unit is positioned based on the location information.

The first microphone, in some aspects, includes a first UWB transceiver coupled thereto and adapted to range with a second UWB transceiver coupled to the locator unit. The computing device is adapted to determine a location of the first microphone based on the ranging between the first and second UWB transceivers.

In some aspects, the system includes a patient support apparatus in communication with the computing device. The patient support apparatus includes a third microphone adapted to output third audio signals representative of sounds detected by the third microphone, and a third transmitter adapted to transmit the third audio signals.

The computing device, in some aspects, is adapted to triangulate a position of the person using the first, second, and third audio signals.

The patient support apparatus, in some aspects, includes a third ultra-wideband (UWB) transceiver adapted to range with the second UWB transceiver, and the computing device is adapted to determine a location of the patient support apparatus based on the ranging between the second and third UWB transceivers.

The patient support apparatus, in some aspects, includes a fourth microphone adapted to output fourth audio signals representative of sounds detected by the fourth microphone, and the third transmitter is adapted to transmit the fourth audio signals.

The computing device, in some aspects, is adapted to triangulate a position of the person using the first, second, third, and fourth audio signals.

The patient support apparatus, in some aspects, includes a plurality of UWB transceivers and a controller adapted to use the plurality of UWB transceivers to determine an orientation of the patient support apparatus relative to a nearby wall in the healthcare facility.

In some aspects, the patient support apparatus includes a memory in which a position of the third microphone on the patient support apparatus is located, the controller adapted to use the third transmitter to transmit the location of the third microphone on the patient support apparatus to the computing device.

The computing device, in some aspects, is adapted to allow a user to select which healthcare facility worker is to receive the warning message.

The computing device, in some aspects, is adapted to detect a pitch and volume of the person's voice when determining the emotional state of the person.

Before the various embodiments disclosed herein are explained in detail, it is to be understood that the claims are not to be limited to the details of operation or to the details of construction and the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus incorporating various aspects of the present disclosure;

FIG. 2 is a plan view of a control panel of the patient support apparatus shown displaying a passcode entry screen;

FIG. 3 is a block diagram of a detailed set of components of the patient support apparatus of FIG. 1, as well as several devices in communication with the patient support apparatus, such as a patient support apparatus server, an electronic device, and a portion of a local area network;

FIG. 4 is a block diagram showing additional details of several servers that may communicate with the patient support apparatus server of FIG. 2;

FIG. 5 is an example of a dashboard screen that may be displayed by one or more display devices in communication with the patient support apparatus server of FIG. 2;

FIG. 6 is a plan view of a motion control screen that may be displayed on the control panel of the patient support apparatus;

FIG. 7 is a plan view of a lock control screen that may be displayed on the control panel of the patient support apparatus;

FIG. 8 is a perspective view of a litter frame and support deck of the patient support apparatus;

FIG. 9 is a perspective view of a seat section of the support deck showing a restraint attachment;

FIG. 10 is a perspective view of the seat section of FIG. 9 showing the restraint attachment covered by a restraint cover;

FIG. 11 is a plan view of an audio monitoring customization screen that may be displayed on the control panel of the patient support apparatus;

FIG. 12 is a plan view of an agitation monitoring customization screen that may be displayed on the control panel of the patient support apparatus;

FIG. 13 is a diagram of a hostile person detection system that may be integrated into one or more of the components described herein; and

FIG. 14 is a perspective view of a video image of an illustrative location within a healthcare facility showing a sound source identifier superimposed upon the image.

### DETAILED DESCRIPTION OF THE EMBODIMENTS

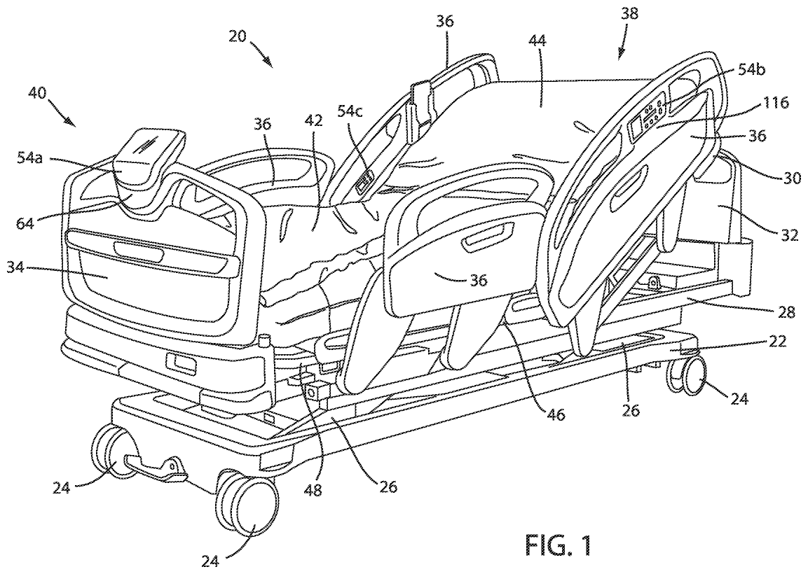

An illustrative patient support apparatus 20 usable in a caregiver assistance system according to the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a recliner, or any other structure capable of supporting a patient while the patient is in a healthcare facility, such as, but not limited to, a hospital and/or a mental health facility. For purposes of the following written description, patient support apparatus 20 will be primarily described as a bed with the understanding that the following written description applies to these other types of patient support apparatuses.

In general, patient support apparatus 20 includes a base 22 having a plurality of wheels 24, a lift subsystem comprising a pair of lifts 26 supported on the base, a litter frame 28 supported on the lifts 26, and a support deck 30 supported on the litter frame 28. Patient support apparatus 20 further includes a headboard 32, a footboard 34, and a plurality of siderails 36. Siderails 36 are all shown in a raised position in FIG. 1 but are each individually movable to a lower position in which ingress into, and egress out of, patient support apparatus 20 is not obstructed by the lowered siderails 36. In some embodiments, siderails 36 may be moved to one or more intermediate positions as well.

Lifts 26 are configured to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted. That is, litter frame 28 includes a head end and a foot end, each of whose height can be independently adjusted by the nearest lift 26. Patient support apparatus 20 is designed so that when an occupant lies thereon, his or her head will be positioned adjacent the head end and his or her feet will be positioned adjacent the foot end. The lifts 26 may be constructed and/or operated in any of the manners disclosed in commonly assigned U.S. patent publication 2017/0246065, filed on Feb. 22, 2017, entitled LIFT ASSEMBLY FOR PATIENT SUPPORT APPARATUS, the complete disclosure of which is hereby incorporated herein by reference. Other manners for constructing and/or operating lifts 26 may, of course, be used.

Litter frame 28 provides a structure for supporting support deck 30, the headboard 32, footboard 34, and siderails 36. Support deck 30 provides a support surface for a mattress 42, or other soft cushion, so that a person may lie and/or sit thereon. Support deck 30 is made of a plurality of sections, some of which are pivotable about generally horizontal pivot axes. In the embodiment shown in FIG. 1, support deck 30 includes at least a head section 44, a seat section 46, and a foot section 48, all of which are positioned underneath mattress 42 and which generally form flat surfaces for supporting mattress 42. Head section 44, which is also sometimes referred to as a Fowler section, is pivotable about a generally horizontal pivot axis between a generally horizontal orientation (not shown in FIG. 1) and a plurality of raised positions (one of which is shown in FIG. 1). Seat section 46 and foot section 48 may also be pivotable about generally horizontal pivot axes.

In some embodiments, patient support apparatus 20 may be modified from what is shown to include one or more components adapted to allow the user to extend the width of patient support deck 30, thereby allowing patient support apparatus 20 to accommodate patients of varying sizes. When so modified, the width of deck 30 may be adjusted sideways in any increments, for example between a first or minimum width, a second or intermediate width, and a third or expanded/maximum width.

As used herein, the term “longitudinal” refers to a direction parallel to an axis between the head end 38 and the foot end 40. The terms “transverse” or “lateral” refer to a direction perpendicular to the longitudinal direction and parallel to a surface on which the patient support apparatus 20 rests.

It will be understood by those skilled in the art that patient support apparatus 20 can be designed with other types of mechanical constructions, such as, but not limited to, that described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another embodiment, the mechanical construction of patient support apparatus 20 may be the same as, or nearly the same as, the mechanical construction of the Model 3002 S3 bed manufactured and sold by Stryker Corporation of Kalamazoo, Michigan. This mechanical construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. It will be understood by those skilled in the art that patient support apparatus 20 can be designed with still other types of mechanical constructions, such as, but not limited to, those described in commonly assigned, U.S. Pat. No. 7,690,059 issued to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The mechanical construction of patient support apparatus 20 may also take on still other forms different from what is disclosed in the aforementioned references.

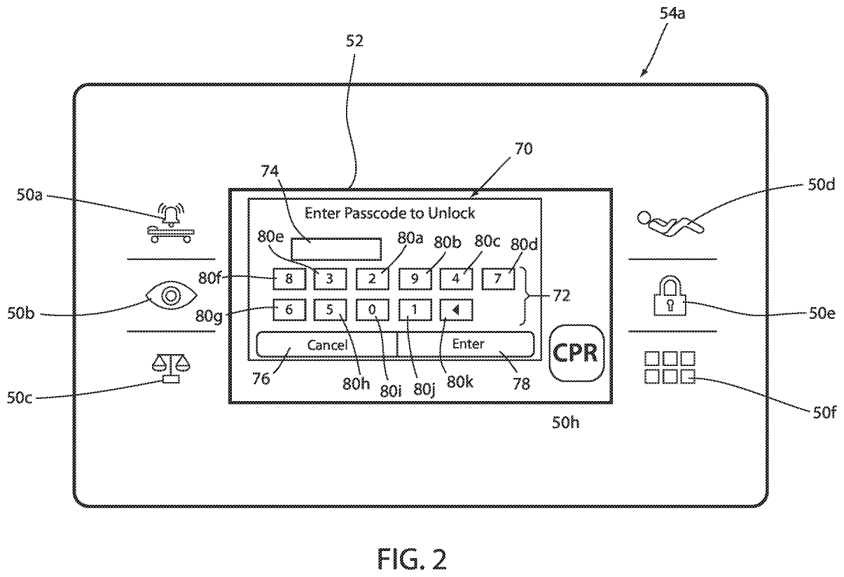

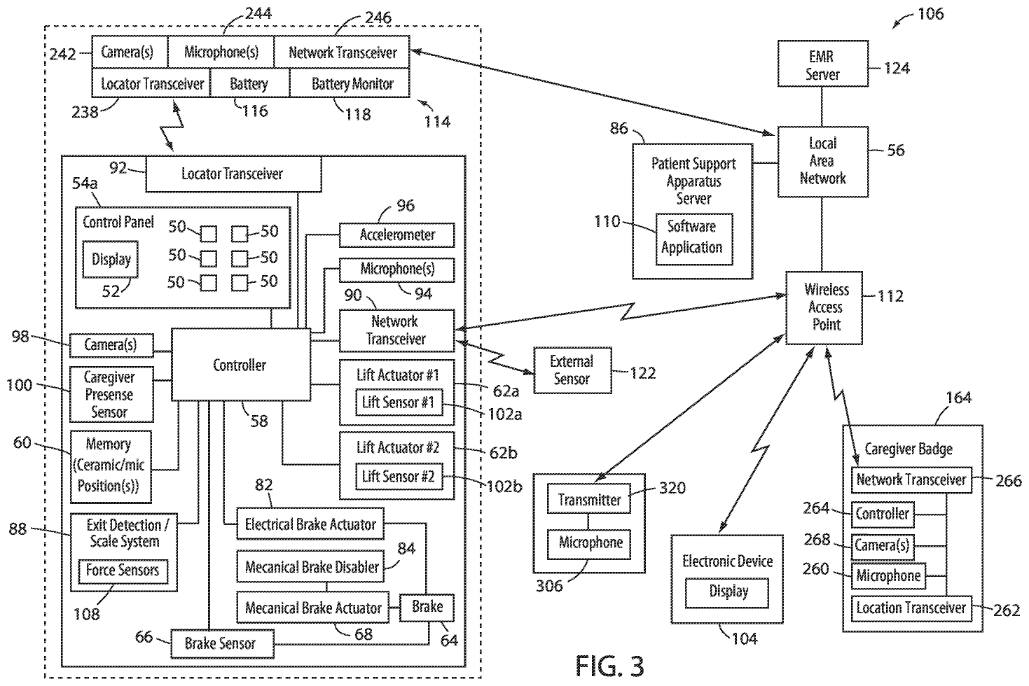

Patient support apparatus 20 further includes a plurality of control panels 54 that enable a user of patient support apparatus 20, such as a patient and/or an associated caregiver, to control one or more aspects of patient support apparatus 20. In the embodiment shown in FIG. 1, patient support apparatus 20 includes a footboard control panel 54a, a pair of outer siderail control panels 54b (only one of which is visible), and a pair of inner siderail control panels 54c (only one of which is visible). Footboard control panel 54a and outer siderail control panels 54b are intended to be used by caregivers, or other authorized personnel, while inner siderail control panels 54c are intended to be used by the patient associated with patient support apparatus 20. Each of the control panels 54 includes a plurality of controls 50 (see, e.g. FIGS. 2-3), although each control panel 54 does not necessarily include the same controls and/or functionality.

Among other functions, controls 50 of control panel 54a allow a user to control one or more of the following: change a height of support deck 30, raise or lower head section 44, activate and deactivate a brake for wheels 24, arm and disarm an exit detection system, activate and deactivate an audio monitor, activate and deactivate an agitation monitor, block and unblock control panel 54a, communicate with the particular IT infrastructure installed in the healthcare facility in which patient support apparatus 20 is positioned, and perform still other functions, some of which are described in greater detail below. One or both of the inner siderail control panels 54c also include at least one nurse-call control that enables a patient to call a remotely located nurse (or other caregiver). In addition to the nurse-call control, one or both of the inner siderail control panels 54c may also include one or more controls for controlling one or more features of a television, room light, and/or reading light positioned within the same room as the patient support apparatus 20. With respect to the television, the features that may be controllable by one or more controls 50 on control panel 54c include, but are not limited to, the volume, the channel, the closed-captioning, and/or the power state of the television. With respect to the room and/or night lights, the features that may be controlled by one or more controls 50 on control panel 54c include the on/off state of these lights.

Control panel 54a includes a display 52 (FIG. 2) configured to display a plurality of different screens thereon. Display 52 may be a touchscreen-type display, although it will be understood that a non-touchscreen display may alternatively be used. Display 52 displays one or more visual indicators, one or more controls, and/or one or more control screens, and/or other types of information, as will be discussed more below. Display 52 may comprise an LED display, an OLED display, or another type of display.

Surrounding display 52 are a plurality of navigation controls 50a-f that, when activated, cause the display 52 to display different screens on display 52. For example, when a user presses navigation control 50a, control panel 54a displays an exit detection control screen on display 52 that includes one or more icons that, when touched, control an onboard exit detection function. The exit detection function is adapted to issue an alert when a patient exits from patient support apparatus 20. Such an exit detection function may include any of the same features and/or functions as, and/or may be constructed in any of the same manners as, the exit detection systems disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES; U.S. patent application Ser. No. 17/318,476 filed May 12, 2021, by inventors Sujay Sukumaran et al. and entitled PATIENT SUPPORT APPARATUS WITH AUTOMATIC EXIT DETECTION MODES OF OPERATION; and/or the exit detection system disclosed in commonly assigned U.S. Pat. No. 5,276,432 issued to Travis and entitled PATIENT EXIT DETECTION MECHANISM FOR HOSPITAL BED, the complete disclosures of all of which are incorporated herein by reference.

When a user presses navigation control 50b (FIG. 2), control panel 54a displays a patient support apparatus monitoring control screen that includes a plurality of control icons that, when touched, control an onboard monitoring system that monitors one or more components, features, and/or other aspects of patient support apparatus 20. Further details of one type of monitoring system that may be built into patient support apparatus 20 are disclosed in commonly assigned U.S. patent application Ser. No. 62/864,638 filed Jun. 21, 2019, by inventors Kurosh Nahavandi et al. and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference. Other types of monitoring systems may be included within patient support apparatus 20 for monitoring parameters of the patient support apparatus 20.

When a user presses navigation control 50c, control panel 54a displays a scale control screen that includes a plurality of control icons that, when touched, control the scale system of patient support apparatus 20. The scale system of patient support apparatus 20 may take on a variety of different forms and include a variety of different features and function. In some embodiments, the scale system may include any of the same features, components, and/or and functions as the scale systems disclosed in the following commonly assigned patent references: U.S. patent application Ser. No. 62/889,254 filed Aug. 20, 2019, by inventors Sujay Sukumaran et al. and entitled PERSON SUPPORT APPARATUS WITH ADJUSTABLE EXIT DETECTION ZONES; U.S. patent application Ser. No. 63/255,211 filed Oct. 13, 2021, by inventors Sujay Sukumaran et al. and entitled PATIENT SUPPORT APPARATUS WITH AUTOMATIC SCALE FUNCTIONALITY; U.S. Pat. No. 10,357,185 issued to Marko Kostic et al. on Jul. 23, 2019, and entitled PERSON SUPPORT APPARATUSES WITH MOTION MONITORING; U.S. Pat. No. 11,33,233 issued to Michael Hayes et al. on Jun. 15, 2021, and entitled PATIENT SUPPORT APPARATUS WITH PATIENT INFORMATION SENSORS; U.S. patent application Ser. No. 16/992,515 filed Aug. 13, 2020, by inventors Kurosh Nahavandi et al. and entitled PATIENT SUPPORT APPARATUS WITH EQUIPMENT WEIGHT LOG; and U.S. patent application Ser. No. 63/255,223, filed Oct. 13, 2021, by inventors Sujay Sukumaran et al. and entitled PATIENT SUPPORT APPARATUS WITH PATIENT WEIGHT MONITORING, the complete disclosures of all of which are incorporated herein by reference. The scale system may utilize the same force sensors that are utilized by the exit detection system, in some embodiments, or it may utilize one or more different sensors.

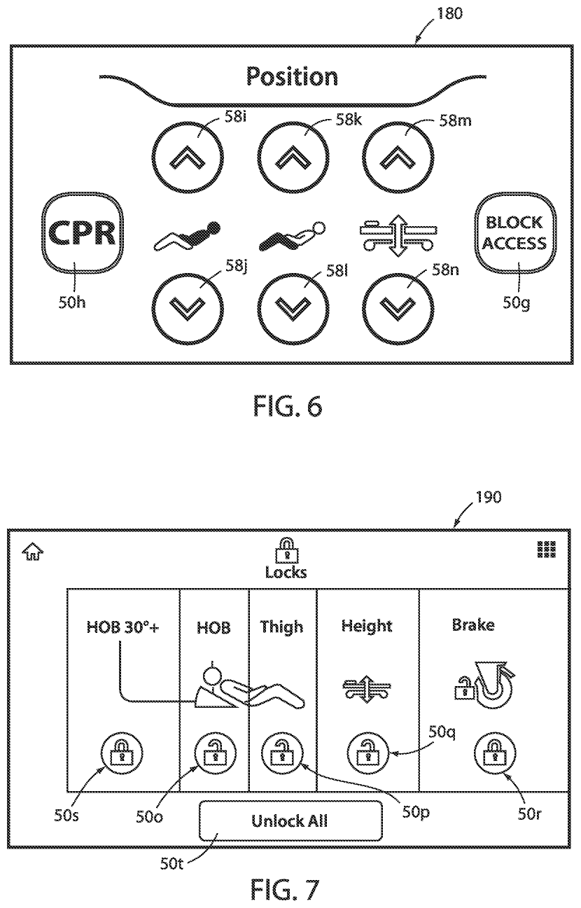

When a user presses navigation control 50d, control panel 54a displays a motion control screen that includes a plurality of control icons that, when touched, control the movement of various components of patient support apparatus 20, such as, but not limited to, the height of litter frame 28 and the pivoting of head section 44. In one embodiment, which will be discussed in greater detail below, patient support apparatus 20 is configured to display a motion control screen of the type shown in FIG. 6 in response to a user pressing on control 50d. In other embodiments, patient support apparatus 20 may be configured to display a motion control screen in response to the user pressing control 50d that may be the same as, or similar to, the motion control screen 216 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of motion control screens may be displayed on display 52 of patient support apparatus 20.

When a user presses navigation control 50e (FIG. 2), control panel 54a displays a motion lock control screen that includes a plurality of control icons that, when touched, control one or more motion lockout functions of patient support apparatus 20. In one embodiment, which will be discussed in greater detail below, patient support apparatus 20 is configured to display a motion lockout screen of the type shown in FIG. 7 in response to a user pressing on control 50e. Such a motion lockout screen may include any of the features and functions as, and/or may be constructed in any of the same manners as, the motion lockout features, functions, and constructions disclosed in commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosure of which is incorporated herein by reference. Other types of motion lockouts may be included within patient support apparatus 20.

In general, pressing on navigation control 50e brings the user to one or more screens that allow the user to selectively disable and enable the functionality of one or more controls on the patient control panels 54c and/or the caregiver siderail control panels 54b. Thus, if a caregiver does not want the patient to be able to move any portions of patient support apparatus 20, he or she may use control 50e to navigate to a lockout screen that enables the caregiver to disable those controls on control panels 54c and/or 54b that control movement of any portions of patient support apparatus 20. In some embodiments, the lockout screen(s) displayed in response to pressing control 50e provide the caregiver with the option for disabling any of the controls on control panels 54b and/or 54c, while in other embodiments, the lockout screens provide the caregiver with the option for disabling only a selected subset of the controls on control panels 54b and/or 54c (such as, but not limited to, the subset of motion controls).

When a user presses on navigation control 50f (FIG. 2), control panel 54a displays a menu screen that includes a plurality of menu icons that, when touched, bring up one or more additional screens for controlling and/or viewing one or more other aspects of patient support apparatus 20. Such other aspects include, but are not limited to, diagnostic and/or service information for patient support apparatus 20, mattress control and/or status information, configuration settings, audio and/or agitation monitoring, location information, and other settings and/or information. One example of a menu screen is the menu screen 100 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of menus and/or settings may be included within patient support apparatus 20.

Control panel 54a is adapted to operate in at least two different modes: a blocked mode and an unblocked mode. In some embodiments, when in the blocked mode, control panel 54a cuts off, or blocks, user-access to a plurality, if not a majority, of the functions of patient support apparatus 20 until the user undertakes a step to verify that they are an authorized user of the patient support apparatus 20, such as entering a password, a passcode, an ID, a biometric input, etc. In such embodiments, control panel 54a may only display a single screen on display 52 (or no screen at all) that displays only a limited set of information and/or controls (such as the time, date, a CPR control 50h, and/or other limited controls and/or data). Control panel 54a prevents the user from navigating on display 52 to all of the other screens, and thus from accessing all of the other functions of patient support apparatus 20 that are controllable on those screens, when control panel 54a is in the blocked mode.

The functions that a user (whether a caregiver, a patient, or another individual) is prevented from accessing when control panel 54a is in the blocked mode include, but are not limited to, the functions that can be accessed through controls 50a-50f. For example, when in the blocked mode, not only can a user not navigate to other screens on display 52, but controls 50a-50f are also inoperative. As a result, the user cannot, for example, control or access the exit detection system, either through pressing on control 50a or through using other navigation controls that may otherwise (when not in the blocked mode) be shown on display 52. Similarly, when control panel 54a is in the blocked mode, the user cannot access or control the monitoring system, either through pressing on control 50b or through using other navigation controls that may otherwise be shown on display 52. When control panel 54a is in the blocked mode, the user also cannot access or control the scale system, motion controls, lockouts, and/or the settings either through pressing on controls 50c-f, respectively, or through using other navigation controls that may otherwise be shown on display 52. Only after the user has entered the correct passcode, password, ID, biometric information, or other information will the control panel 54a allow the user to access these and other functions.

It will be understood that, in some embodiments, the blocked mode of footboard control panel 54a does not automatically cause any of the other control panels 54b and/or 54c to operate in a blocked mode. That is, in some embodiments, control panels 54b and/or 54c do not operate in a blocked mode when control panel 54a operates in the blocked mode. However, it will be understood that, if one of the lockout functions of control panel 54a (accessed via control 50e) has been activated such that one or more controls on control panels 54b and/or 54c are disabled, those controls will remain disabled while control panel 54a is in the blocked mode (as well as while in the unblocked mode). As a consequence, if a caregiver doesn't want a patient to be able to use a motion control on a patient control panel 54c, for example, he or she can lock out that control using lockout control 50e (while control panel 54a is in the unblocked mode) and then, after control panel 54c enters the blocked mode, it will be impossible for the patient to unlock the locked out control unless he or she knows the passcode, password, or other ID that must be entered in order to change control panel 54a from the blocked mode to the unblocked mode. In this manner, caregivers can rest assured that, once they lock out a motion control (or make other changes using control panel 54a) and control panel 54a enters the blocked mode, the patient will not be able to use the motion control (or otherwise have any access to the function accessible via control panel 54a) until an authorized person returns to patient support apparatus 20 and enters the correct password, passcode, ID, etc.

It will also be understood that the siderail control panels 54b and 54c, in at least some embodiments, don't offer access to the same functions and/or controls that control panel 54a does. Thus, even if these control panels remain in an unblocked mode while control panel 54a is in the blocked mode, the patient (or another person) is not able to use these control panels 54b and/or 54c to access the functions that are accessible via control panel 54a (after the correct password, passcode, ID, etc. has been entered). In some embodiments, control panels 54b and 54c include the same functionality as the control panels 44b and 44c disclosed in commonly assigned U.S. patent application (P-671A) Ser. No. 63/417,516 filed Oct. 19, 2022, by inventors Sujay Sukumaran et al. and entitled PATIENT SUPPORT APPARATUS WITH LOCKING FEATURES, the complete disclosure of which is incorporated herein by reference. Other types of siderail control panels 54b and/or 54c may, of course, be used.

In some embodiments, control panel 54a is configured to automatically switch to the blocked mode in response to a predetermined amount of time passing without usage of control panel 54a, such as a minute or so. In other words, control panel 54a is configured to operate in the unblocked mode for only a window of time. The window of time may be a static amount of time measured from the moment the correct passcode is successfully entered, and this window of time may be automatically extended each time the control panel 54a is used while in the unblocked mode. As a result, the window of time will expire either after a static amount of time passes since the correct passcode is entered (if no controls on control panel 54a are thereafter activated), or a static amount of time passes since the user last activated a control on control panel 54a). Stated alternatively, control panel 54a is configured to automatically switch to the blocked mode after a period of non-use (the window of time).

In some embodiments, control panel 54a may also, or alternatively, be configured to allow the user to manually switch from the unblocked mode to the blocked mode. In such embodiments, patient support apparatus 20 includes a block control, such as the block access control 50g shown in FIG. 6. In response to a user activating block control 50g, the control panel 54a switches to the blocked mode and displays a passcode screen, such as the passcode screen 70 shown in FIG. 2. Access to the normal functionality of control panel 54a is thereafter blocked until the user enters the correct passcode.

Passcode screen 70 (FIG. 2) includes a key pad 72, a code entry field 74, a cancel control 76, an enter control 78, and a plurality of keys 80a-k. In order for a user to enter a passcode, the caregiver presses on a series of the keys 80a-j. For each key 80a-j that is pressed by the caregiver, control panel 54a displays the number corresponding to the pressed key 80a-j in code entry field 74. If the user presses on an inadvertent key, the user can erase the numeric entry by pressing on the backspace key 80k, which, for each press of key 80k, erases the most recently entered number in field 74. Once the desired passcode has been entered into field 74, the user presses on enter control 78 and a controller (not shown in FIG. 2) checks to see if the code entered in field 74 matches the code stored in the memory of patient support apparatus 20. If they match, the controller unblocks control panel 54a, such as by displaying the screen shown in FIG. 6 (or another screen) on display 52. If they do not match, the controller continues to block access to the functionality of control panel 54a, thereby preventing unauthorized usage of the control panel 54a (such as by the patient). If the caregiver presses the cancel control 76, the controller continues to block access to the full usage of control panel 54a.

In order to help prevent a patient from deducing the correct passcode by watching the movement patterns of the caregiver's hand and/or fingers while he or she enters the correct passcode (and/or to help prevent the patient from guessing the passcode through a fingerprint smudge analysis), the controller may be configured to scramble the numbers assigned to keys 80a-j each time it displays a passcode screen 70. By doing this, each time a caregiver enters the correct passcode, he or she will utilize a different hand movement pattern (and press different areas on touchscreen display 52), thereby making it more difficult for a patient who is watching the caregiver (but doesn't see screen 70) to determine what the correct passcode is. Further details of one of example of this type of key scrambling is disclosed in commonly assigned U.S. patent application Ser. No. 63/417,516 filed Oct. 19, 2022, by inventors Sujay Sukumaran et al. and entitled PATIENT SUPPORT APPARATUS WITH LOCKING FEATURES, the complete disclosure of which is incorporated herein by reference. Of course, in some embodiments, key pad 72 may be displayed without scrambling the keys 80, while in still other embodiments, keys 80 may be scrambled in manners different from those disclosed in the aforementioned patent application.

As was noted above, when control panel 54a is in the blocked state, it prevents anyone from accessing a majority of the functions that are controllable by control panel 54a unless they enter the correct passcode. In some embodiments, patient support apparatus 20 may be configured to allow a user to still access at least one function via control panel 54a, even when control panel 54a is in the blocked state. For example, in at least one embodiment, control panel 54a includes a CPR control 50h (FIG. 2) that is displayed on display 52 and that (when display 52 is a touch screen), when pressed, activates a CPR function for patient support apparatus 20. The CPR function automatically lowers head section 44 to a generally horizontal orientation so that a healthcare worker can more easily perform CPR on a patient lying on patient support apparatus 20. In some embodiments, activation of the CPR function may also automatically flatten seat section 46 and foot section 48 and/or activate lifts 26 such that litter frame 28 is brought to a horizontal orientation and a particular height. Still further, in some embodiments, patient support apparatus 20 may be configured to place the CPR control 50h on a screen of display 52 that can only be accessed after the correct passcode has been entered. In other words, in some embodiments, patient support apparatus 20 is modified from what is shown in FIG. 2 and access to CPR control 50h is blocked when control panel 54a is in the blocked mode.

In some embodiments, one of the functions that a user is blocked from accessing when control panel 54a is in the blocked mode is a brake for patient support apparatus 20. In such embodiments, patient support apparatus 20 includes an electrically activated and deactivated brake that can only be controlled via control panel 54a when control panel 54a is in the unblocked mode. In addition, in such embodiments, patient support apparatus 20 may include a mechanical brake that can be electrically disabled via control panel 54a when control panel 54a is in the unblocked mode. In this manner, if the caregiver does not want the patient to be able to change the brake status of patient support apparatus 20, the caregiver can set the brake to the desired state (braked or unbraked) using control panel 54a (while in the unblocked mode), and then use control panel 54a to disable the mechanical brake. In this manner, once the control panel 54a enters its blocked mode, the state of the brake cannot be changed without the correct passcode.

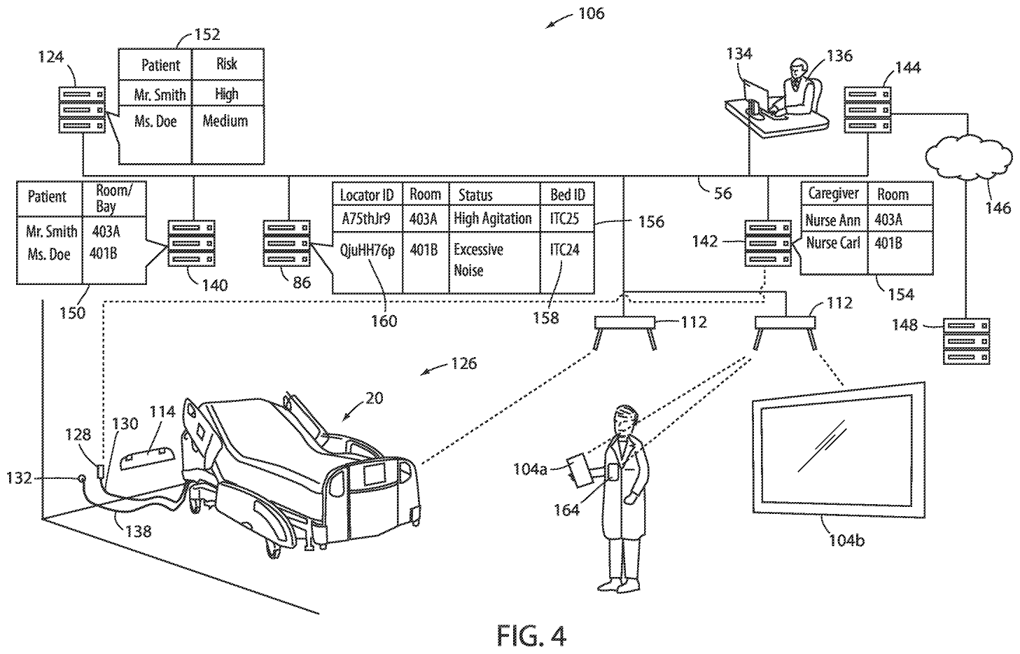

FIG. 3 illustrates a first embodiment of a caregiver assistance system 106 according to the present disclosure. Caregiver assistance system 106 includes patient support apparatus 20 in communication with a patient support apparatus server 86, and one or more display devices 104 that are adapted to communicate with patient support apparatus server 86. The patient support apparatus server 86, like all of the servers discussed herein, includes one or more conventional microprocessors. Patient support apparatus server 86 is adapted to execute a software application 110 that receives various data from one or more patient support apparatuses 20 and forwards some, or all, of this data to one or more display devices 104 for display thereon. As will be discussed in greater detail below with respect to FIG. 4, software application 110 may communicate with a plurality of other servers on a local area network 56 of the healthcare facility and use those communications to obtain some of the information it needs to perform some of the caregiver assistance functions described herein.

FIG. 2 illustrates in greater detail some of the internal components of patient support apparatus 20. As shown therein, patient support apparatus 20 includes a controller 58, a memory 60, a first lift actuator 62a, a second lift actuator 62b, a brake 64, a brake sensor 102 a mechanical brake actuator 68, an electrical brake actuator 82, a mechanical brake disabler 84, a scale/exit detection system 88, control panel 54a (as well as control panels 54b and 54c, which are not shown in FIG. 3), a network transceiver 90, a location transceiver 92, a microphone 94, an accelerometer 96, a camera 98, a caregiver presence detector 100, and first and second lift sensors 102a and 102b. Additionally, control panel 54a includes display 52 and controls 50, while exit detection system 88 includes a plurality of force sensors 108. It will be understood by those skilled in the art that patient support apparatus 20 may be modified to include additional components not shown in FIG. 3, as well modified to include fewer components from what is shown in FIG. 2.

Controller 58 (FIG. 3) is constructed of any electrical component, or group of electrical components, that are capable of carrying out the functions described herein. In many embodiments, controller 58 is a conventional microcontroller, or group of conventional microcontrollers, although not all such embodiments need include a microcontroller. In general, controller 58 includes any one or more microprocessors, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units as part of an embedded network. When implemented to include an embedded network, the embedded network may include multiple nodes that communicate using one or more of the following: a Controller Area Network (CAN); a Local Interconnect Network (LIN); an I-squared-C serial communications bus; a serial peripheral interface (SPI) communications bus; any of RS-232, RS-422, and/or RS-485 communication interfaces; a LonWorks network, and/or an Ethemet. The instructions followed by controller 58 in carrying out the functions described herein, as well as the data necessary for carrying out these functions, are stored in memory 60, and/or in one or more other memories accessible to the one or more microprocessors, microcontrollers, or other programmable components of controller 58. Memory 60 also includes a unique identifier that uniquely identifies the particular patient support apparatus into which it is incorporated, such as, but not limited to, a serial number.

First and second lift actuators 62a and 52b (FIG. 3) are components of lifts 26 and are configured to raise and lower litter frame 28 with respect to base 22. A first one of lift actuators 62a powers a first one of the lifts 26 positioned adjacent head end 38 of patient support apparatus 20 and a second one of lift actuators 62b powers a second one of the lifts 26 positioned adjacent foot end 40 of patient support apparatus 20. Lift actuators 62a and 62b may be conventional linear actuators having electric motors therein that, when driven, expand or contract the length of the linear actuator, thereby moving the litter frame upward or downward and changing its height relative to the floor.

Each lift actuator 62a and 62b includes a corresponding lift sensor 102a and 102b, respectively. Each of the sensors 102a, 102b detects a position and/or angle of its associated actuator 62a, 62b and feeds the sensed position/angle to controller 58. Controller 58 uses the outputs from sensors 102 as inputs into a closed-loop feedback system for controlling the motion of the actuators 62a, 62b and the litter deck. Controller 58 also uses the outputs from sensors 102a, 102b to determine the height of litter frame 28 above the floor. In some embodiments, actuators 62 are constructed in any of the same manners as the actuators 34 disclosed in commonly assigned U.S. patent application Ser. No. 15/449,277 filed Mar. 3, 2017, by inventors Anish Paul et al. and entitled PATIENT SUPPORT APPARATUS WITH ACTUATOR FEEDBACK, the complete disclosure of which is incorporated herein by reference. In such embodiments, sensors 102a and 102b may be constructed to include any of the encoders and/or switch sensors disclosed in the aforementioned '277 application.

Scale/exit detection system 88 is configured to determine a weight of a patient positioned on support deck 30 and/or when the patient is moving and is likely to exit patient support apparatus 20. The particular structural details of the exit detection system can vary widely. In some embodiments, scale/exit detection system 88 includes a plurality of load cells 108 arranged to detect the weight exerted on litter frame 28. By summing the outputs from each of the load cells 108, the total weight of the patient is determined (after subtracting the tare weight). Further, by using the known position of each of the load cells 108, controller 58 determines a center of gravity of the patient and monitors the center of gravity for movement beyond one or more thresholds. One method of computing the patient's center of gravity from the output of such load cells is described in more detail in commonly assigned U.S. Pat. No. 5,276,432 issued to Travis and entitled PATIENT EXIT DETECTION MECHANISM FOR HOSPITAL BED, the complete disclosure of which is incorporated herein by reference. Other methods by which scale/exit detection system 88 may be implemented in order to determine when a patient is likely to exit from patient support apparatus 20 are disclosed in commonly assigned U.S. patent application Ser. No. 17/318,476 filed May 12, 2021, by inventors Sujay Sukumaran et al. and entitled PATIENT SUPPORT APPARATUS WITH EXIT DETECTION MODES OF OPERATION, the complete disclosure of which is incorporated herein by reference. Still other methods of detecting when a patient has exited, or is about to exit, from patient support apparatus 20 may be implemented by scale/exit detection system 88.

Scale/exit detection system 88 may also implement one or more other methods for determining a patient's weight and/or the weight of non-patient objects supported on litter frame 28, such as any of the methods and/or structures that are disclosed in commonly assigned U.S. patent application Ser. No. 14/776,842, filed Sep. 15, 2015, by inventors Michael Hayes et al. and entitled PATIENT SUPPORT APPARATUS WITH PATIENT INFORMATION SENSORS, and commonly assigned U.S. patent application Ser. No. 14/873,734 filed Oct. 2, 2015, by inventors Marko Kostic et al. and entitled PATIENT SUPPORT APPARATUSES WITH MOTION MONITORING, the complete disclosures of both of which are incorporated herein by reference. Scale/exit detection system 88 may utilize still other methods and/or structures for determining a patient's weight.

In some embodiments, mattress 42 (FIG. 1) is an inflatable mattress. In such embodiments, mattress 42 may include its own internal controller (not shown) that controls the inflation and deflation of various bladders contained within mattress under the instructions of controller 58. In other embodiments, controller 58 may directly control the blower(s), pump(s), valve(s), and other components of mattress 42. In either situation, controller 58 may communicate with mattress 42 using a serial cable, or other cable, that extends between patient support apparatus 20 and mattress 42. In at least one alternative embodiment, the communication between patient support apparatus 20 and mattress 42 may be carried out wirelessly, such as in any of the manners disclosed in commonly assigned U.S. Pat. No. 9,289,336 issued to Lambarth et al. and entitled PATIENT SUPPORT WITH ENERGY TRANSFER, the complete disclosure of which is incorporated herein by reference. Other manners for wireless communication may, of course, be used.

Controller 58 communicates with network transceiver 90 (FIG. 3) which, in at least one embodiment, is a Wi-Fi radio communication module configured to wirelessly communicate with wireless access points 112 of local area network 56. In such embodiments, network transceiver 90 may operate in accordance with any of the various IEEE 802.11 standards (e.g. 802.11b, 802.11n, 802.11g, 802.11ac, 802.11ah, etc.). In other embodiments, network transceiver 90 may include, either additionally or in lieu of the Wi-Fi radio and communication module, a wired port for connecting a network wire to patient support apparatus 20. In some such embodiments, the wired port accepts a category 5e cable (Cat-5e), a category 6 or 6a (Cat-6 or Cat-6a), a category 7 (Cat-7) cable, or some similar network cable, and transceiver 90 is an Ethernet transceiver. In still other embodiments, network transceiver 90 may be constructed to include the functionality of the communication modules 56 disclosed in commonly assigned U.S. patent application Ser. No. 15/831,466 filed Dec. 5, 2017, by inventor Michael Hayes et al. and entitled NETWORK COMMUNICATION FOR PATIENT SUPPORT APPARATUSES, the complete disclosure of which is incorporated herein by reference.

Regardless of the specific structure included with network transceiver 90, controller 58 is able to communicate with the local area network 56 (FIG. 3) of a healthcare facility in which the patient support apparatus is positioned. When network transceiver 90 is a wireless transceiver, it communicates with local area network 56 via one or more wireless access points 112. When network transceiver 90 is a wired transceiver, it communicates directly via a cable coupled between patient support apparatus 20 and a network outlet positioned within the room of the healthcare facility in which patient support apparatus 20 is positioned. As will be discussed in greater detail below with respect to FIG. 4, local area network 56 includes a plurality of servers that are utilized in different manners by the caregiver assistance system 106 disclosed herein, and patient support apparatus 20 communicates with one or more of those servers (e.g. patient support apparatus server 86) via transceiver 90 as part of the caregiver assistance system.

In some embodiments, patient support apparatus 20 may include a nurse call cable interface (not shown) that is adapted to couple to one end of a conventional nurse call cable 130 (FIG. 4). The other end of the nurse call cable couples to a nurse call outlet 128 that is typically built into each headwall of each of the patient rooms within a healthcare facility. In many embodiments, the nurse call outlet is a 37 pin outlet that the cable couples to, thereby enabling patient support apparatus 20 to communicate directly with a conventional nurse call system. In some embodiments, the nurse call cable interface of patient support apparatus 20 is constructed in accordance with any of the cable interfaces 92 disclosed in commonly assigned U.S. patent application Ser. No. 15/945,437 filed Apr. 4, 2018, by inventors Krishna Bhimavarapu et al. and entitled PATIENT SUPPORT APPARATUSES WITH RECONFIGURABLE COMMUNICATION, the complete disclosure of which is incorporated herein by reference.

In other embodiments, the nurse call cable interface may be replaced with a wireless nurse call communication system that wirelessly communicates with the nurse call outlet 128. For example, in some embodiments, the nurse call cable interface may be replaced with a radio module, such as the radio module 60 disclosed in commonly assigned U.S. patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION, the complete disclosure of which is incorporated herein by reference. In such wireless headwall embodiments, a headwall module, such as headwall module 38 disclosed in the aforementioned '844 application, is included and coupled to the nurse call outlet. Such a headwall module may replace and/or supplement the functions of location beacon 114, described below. In some embodiments, the nurse call interface may also, or alternatively, perform any of the functions of the nurse call interfaces disclosed in commonly assigned U.S. patent application Ser. No. 62/833,943 filed Apr. 15, 2019, by inventors Alexander Bodurka et al. and entitled PATIENT SUPPORT APPARATUSES WITH NURSE CALL AUDIO MANAGEMENT, the complete disclosure of which is also incorporated herein by reference. Still other types of wireless communication between the patient support apparatus and a nurse call outlet 128 may be implemented.

Location transceiver 92 (FIG. 3) is adapted to detect a wireless signal emitted from a nearby location beacon 114 that is positioned at a fixed and known location within the healthcare facility. Although FIG. 3 only illustrates a single one of these location beacons 114, it will be understood that a particular healthcare facility includes many of these location beacons 114 mounted throughout the healthcare facility. Each location beacon 114 includes a wireless short range transmitter (not shown) that broadcasts a wireless, short range signal containing a unique identifier. The short range signal, in some embodiments, is broadcast via an infrared transmitter and is only detectable by receivers (e.g. location transceivers 92) that are positioned within several feet of the location beacon 114. Consequently, location transceivers 92, which are adapted to detect the signals transmitted from location beacons 114, are only able to detect these signals when patient support apparatuses 20 are positioned adjacent (e.g. within several feet) of one of these location beacons 114. If/when location transceiver 92 is able to detect the unique signal from a particular location beacon 114, the corresponding patient support apparatus 20 can therefore be concluded to be currently positioned adjacent that particular location beacon 114. This allows the current location of the patient support apparatus 20 to be identified. In some healthcare facilities, one or more of the patient rooms may not be completely private rooms, but instead may be shared with one or more other patients. In such situations, it is typical to mount two or more location beacons 114 within such a room-one on the headwall at the bay where the first patient support apparatus 20 normally resides and the other on the headwall at the bay where the second patient support apparatus 20 normally resides (and still more if the room is shared by more than two patients).

In some embodiments of patient support apparatus 20, location transceiver 92 may be an ultra-wideband (UWB) transceiver adapted to receive and/or transmit UWB signals. When so implemented, location transceiver 92 may be able to use UWB signals to communicate with location transceiver 238 that is also a UWB transceiver. By exchanging UWB signals between themselves (e.g. ranging), location transceivers 92 and 238 are able to determine their distance from each other. In some embodiments, patient support apparatus 20 may include multiple location transceivers 92 positioned at known locations onboard patient support apparatus 20 and use ranging between those multiple UWB transceivers 92 and the UWB transceiver(s) 238 on location beacon 114 to determine the orientation of patient support apparatus 20 relative to location beacon 114, the wall to which it is attached, and/or the room in which the locator unit 114 is positioned. In some embodiments, locator units 114 and/or patient support apparatus 20 may include any of the same UWB functionality as the locator units 60 and/or patient support apparatuses 20 disclosed in commonly assigned U.S. provisional patent application Ser. No. 63/597,412 filed Nov. 9, 2023, by inventors Michael Graves et al. and entitled PATIENT SUPPORT APPARATUS WITH ENVIRONMENTAL INTERACTION, the complete disclosure of which is incorporated herein by reference.

When location transceiver 92 receives a signal from an adjacent location beacon 114, controller 58 forwards the received signal, including the unique ID 160 of the beacon 114 and a unique ID 158 of patient support apparatus 20 to software application 110 of patient support apparatus server 86 (FIG. 4). Software application 110 includes and/or utilizes a table that correlates beacon IDs to locations (e.g. rooms) within the healthcare facility. Software application 110 is thereby able to determine the location of each patient support apparatus 20 within the healthcare facility (at least all of those that are positioned adjacent a location beacon 114).

In some embodiments, location beacons 114 (FIG. 3) function both as locators and as wireless links to a nurse call outlet 128 (FIG. 4) integrated into the adjacent headwall. When equipped with this dual function, patient support apparatuses 20 may omit the aforementioned nurse call cable interface, yet still be able to communicate with the nurse call system. Further details about the function of location beacons 114, whether operating solely as locators or both as locators and wireless portals to the nurse call system outlets, may be found in any of the following commonly assigned U.S. patent references: U.S. Pat. No. 8,102,254 issued Jan. 24, 2012 to Becker et al. and entitled LOCATION DETECTION SYSTEM FOR A PATIENT HANDLING DEVICE; patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION; patent application Ser. No. 62/600,000 filed Dec. 18, 2017, by inventor Alex Bodurka, and entitled SMART HOSPITAL HEADWALL SYSTEM; and patent application Ser. No. 62/598,787 filed Dec. 14, 2017, by inventors Alex Bodurka et al. and entitled HOSPITAL HEADWALL COMMUNICATION SYSTEM, the complete disclosures of all of which are incorporated herein by reference.

Location beacon 114 also includes, in at least some embodiments, a beacon battery 116 and a beacon battery monitor 118 (FIG. 3). Beacon battery 116 provide electrical power to location beacon 114, either exclusively or, in at least some embodiments, when location beacon 114 is unplugged, or electrical power is otherwise unavailable from an electrical outlet. Beacon battery monitor 118 monitors the charge state of beacon battery 116 and reports measurements of this charge to patient support apparatus 20. That is, the measurements taken by beacon battery monitor 118 are forwarded wirelessly by location beacon 114 to patient support apparatus 20 via the built-in transmitter of location beacon 114. These measurements are received by location transceiver 92 onboard patient support apparatus 20 and forwarded to controller 58. Controller 58 then displays these measurements on display 52 and/or forwards them to software application 110 via network transceiver 90. Software application 110 may forward these battery charge measurements to one or more display devices 104 for display thereon.

In some embodiments, beacon battery monitor 118 may monitor one or more additional factors regarding beacon battery 116, such as, but not limited to, the overall health of beacon battery 116. Such overall health may be measured in terms of the charge capacity of the battery, the number of times the battery has been recharged, the rate at which the battery discharges, the rate at which the battery re-charges, and/or in other manners. In some embodiments, beacon battery monitor 118 may be implemented in the same manner as, and/or configured to monitor and measure any one or more of the same battery parameters as, the battery monitors disclosed in commonly assigned U.S. patent publication 2016/0331614 published Nov. 17, 2016, and filed by inventors Aaron Furman et al. and entitled BATTERY MANAGEMENT FOR PATIENT SUPPORT APPARATUSES, the complete disclosure of which is incorporated herein by reference.

In some embodiments, location beacon 114 may be incorporated into a wireless headwall module that communicates with patient support apparatus 20 over multiple communication channels. In such embodiments, the first communication channel between location beacon 114 and patient support apparatus 20 may be a short range channel (e.g. infrared) and the second one may be a longer range channel (e.g. Bluetooth). In such embodiments, the transmission of the data from beacon battery monitor 118 to patient support apparatus 20, as well as the transmission of the location identifier of location beacon 114 to patient support apparatus 20, may occur over either or both of the two communication channels.

In some embodiments, location beacon 114 may also include one or more cameras 242, one or more microphones 244, and a network transceiver 246. Microphone(s) 244 are adapted to capture sounds emitted in the vicinity of location beacon 114 and camera(s) 242 are adapted to capture video images of the areas surrounding location beacon 114, such as the area inside of a room or hallway in which the location beacon 114 is positioned. Network transceiver 246 is adapted to allow location beacon 114 to communicate with local network 56. Network transceiver 246 may be the same type of transceiver as the network transceiver 90 onboard patient support apparatus 20. As will be discussed in greater detail below, when location beacon 114 includes one or more cameras 242, microphones 244, and at least one transmitter (e.g. network transceiver 246 and/or location transceiver 238), location beacon 114 may form part of a hostile person detection system 300 that is adapted to automatically detect potentially hostile individuals within an area of a healthcare facility.

Patient support apparatus 20 includes one or more cameras 98. Camera(s) 98 (and/or camera(s) 242), in several embodiments, are video cameras adapted to capture moving images. However, in some other embodiments, camera(s) 98 (and/or camera(s) 242) may be still-image cameras, thermal image cameras (still or video), or other types of cameras. Camera(s) 98 are aimed and/or have fields of view that allow the camera(s) 98 to capture images of not only patient support apparatus 20, but also the areas surrounding patient support apparatus 20. As will be discussed in greater detail below, camera(s) may be adapted to perform one or more of the following tasks: capture images of the patient in order to determine an agitation level of the patient; capture images of the area surrounding patient support apparatus 20 in order to determine whether a caregiver is currently positioned within the vicinity of the patient support apparatus 20 (e.g. within the same room); capture images of the patient to detect rapid movement of the patient, the throwing of items by the patient, and/or or other unwanted behaviors of the patient; capture images of the patient to detect whether the patient is positioned onboard patient support apparatus 20 or offboard patient support apparatus 20; capture images of patient support apparatus 20 and/or its surroundings in order to determine whether patient support apparatus 20 is moving; capture images of the patient to determine whether the patient is absent from patient support apparatus 20 for more than a set period of time; capture images of the patient in order to determine the position and/or orientation of the patient, such as, but not limited to, if the patient is upside-down on patient support apparatus 20 (i.e. patient's head is near foot end 40 of patient support apparatus 20), if the patient has become entrapped between a siderail 36 and the support deck 30 and/or mattress 42, and/or if the patient is interacting with patient support apparatus 20 and/or other items or equipment within the same room; to capture images of the patient in order to determine if the patient is tampering with any items that could be used as a ligature for harming the patient or others; and/or capture other images of the patient's behavior in order to assess a level of risk of self-harm and/or harm to others that the patient may demonstrate.

In some embodiments, one or more of camera(s) 98 may include infrared, or other thermal imaging capabilities. Such thermal imaging capabilities may be used to capture thermal images that allow controller 58 to detect if blood, urine, or fecal matter has been excreted by the patient; to detect if a patient has repeatedly rubbed an object against concrete, metal, or another object in order to sharpen the object (which would lead to an increased temperature detectable by the thermal images); to detect if the patient has lit anything on fire within the room; and/or to detect other behavior by the patient that may be destructive or indicative of an intent to destroy property and/or hurt themselves or others. The processing of the images captured by camera(s) 98, whether thermal images and/or visible light images, is performed by controller 58, and/or one or more other controllers that are in communication with controller 58.

In some embodiments, camera(s) 98 may include any of the same features, functions, locations, and/or other characteristics of the cameras disclosed in commonly assigned U.S. patent application Ser. No. 63/218,053 filed Jul. 2, 2021, by inventors Krishna Bhimavarapu et al. and entitled PATIENT VIDEO MONITORING SYSTEM, the complete disclosure of which is incorporated herein by reference. Still other types of cameras may also, or alternatively, be used. In some embodiments, one or more cameras 98 may be used as part of caregiver assistance system 106 that are not mounted to patient support apparatus 20, but instead are mounted to the wall, ceiling, or other locations.

Patient support apparatus 20 further includes one or more accelerometers 96 (FIG. 3). Controller 58 is in communication with the accelerometer(s) 96 and uses the outputs of the accelerometer(s) 96 to determine whether patient support apparatus 20 is in motion or not (i.e. whether patient support apparatus 20 is being rolled on wheels 24, or otherwise moved, across the floor. Accelerometer(s) 96 may therefore be placed at any suitable location on patient support apparatus 20 that will experience accelerations when patient support apparatus 20 is moved in such a manner. Accelerometer(s) 96 are configured to detect movement in at least two dimensions (forward-backward and side-to-side). In some embodiments, accelerometer(s) 96 may be configured to also detect movement of patient support apparatus 20 in a third dimension (up-down). The outputs of the accelerometer(s) 96 are reported to controller 58 so that controller 58 may take appropriate action(s) in response to detecting movement of patient support apparatus 20, particularly when no caregiver is positioned in the room and/or at other times, as will be discussed in more detail below. And, as will also be discussed more below, controller 58 may use the movement of patient support apparatus 20 when determining an agitation level of the patient and/or when assessing a level of risk of self-harm and/or harm to others that the patient may be demonstrating.

In some embodiments, controller 58 is configured to use the outputs from accelerometer(s) 96, either alone or in combination with the force sensors 108, to detect one or more of the following: (a) rapid movement of the patient while on patient support apparatus 20 (such as indicated through rapid increases/decreases in the net weight detected by force sensors 108, through rapid transfer of forces from one force sensor 108 to another, through rapid changes in the patient's center of gravity, etc.); (b) the patient frequently getting into and out of patient support apparatus 20, particularly during evening hours; (c) the patient moving to a position indicating they intend to exit from patient support apparatus 20; (d) decreases in the net weight supported by patient support apparatus 20 (which are indicative of items being removed from patient support apparatus 20); (e) negative or decreased gross weight detected by force sensors 108 which may indicate, either alone or in combination with outputs from accelerometer(s) 96, that patient support apparatus 20 has been flipped on its side or is positioned upside down; (f) a load applied to the perimeter of frame 28 and/or support deck 30 (which may be indicative of a ligature or the patient otherwise using the frame 28 and/or support deck 30 to injure themselves); (g) impacts that the patient support apparatus 20 may make with a wall or other obstruction; (h) impacts made in the surroundings that generate vibrations detectable by force sensors 108 and/or accelerometer(s) 96 (e.g. a patient jumping up and down near patient support apparatus, equipment being dropped, etc.); (i) movement of patient support apparatus 20 across the floor while a caregiver is not in the room (which may indicate the brake 64 of patient support apparatus 20 has been compromised or the bed has been flipped, slid, or otherwise moved by the patient); (j) changes in the orientation of patient support apparatus 20; and/or (h) one or more vital signs of the patient, such as heart rate, respiration rate, etc.

Patient support apparatus 20 further includes one or more microphones 94 that are adapted to capture sounds within the room in which patient support apparatus 20 is positioned. In some embodiments, microphone 94 may the same microphone that the patient speaks into when he or she calls a remotely positioned nurse, in which case the voice signals detected by microphone 94 are converted to audio signals and forwarded to the nurse call system (which in turn routes them to the appropriate nurses' station). In other embodiments, one or more microphones 94 may be included that are separate from, and/or in addition to, the microphone used by the patient to talk to a remotely positioned nurse. Regardless of whether or not one or more of microphone(s) 94 are used for communicating audio signals to a nurse call system, microphone(s) 94 are used in conjunction with controller 58 to perform any one or more of the following functions: detect noise levels within the vicinity of patient support apparatus 20 (e.g. within the room in which patient support apparatus 20 is located; perform a speech recognition function that detects key words or key phrases uttered by the patient that may indicate the patient has some intent to harm themself and/or another person (e.g. “kill myself,” “suicide,” “die,” “hang,” “suffocate,” “cut,” etc.); and/or perform a speech recognition function that detect key words and/or phrases uttered by the patient that may indicate the patient is agitated (e.g. “exhausted,” “tired,” “mad,” “hate,” “attack,” “annoyed,” “run away,” and/or profanity).

In some embodiments, controller 58 may be configured to record a clip of any audio event. That is, in some embodiments, controller 58 may records the noises and/or speech that it detects and, if a noise is detected above a threshold and/or if one or more key words and/or key phrase are detected, controller 58 may capture a segment or clip of the recorded audio that includes both the moments after, and the moments before, the noise, key word, and/or key phrase. Such audio clips are forwarded by controller 58, in some embodiments, to patient support apparatus server 86 which, as discussed further, may be configured to send the audio clips, and/or other information about the audio clips, to one or more display devices 104. In those embodiments of system 106 in which one or more cameras 98 are installed, controller 58 may also, or alternatively, send one of more video clips from the camera(s) 98 to patient support apparatus server 86 in response to a microphone 94 detecting a noise above a threshold, a key word, and/or a key phrase. Alternatively, or additionally, controller 58 may send a video clip of camera(s) 98 to patient support apparatus server 86 in response to the detection of any one or more of the events discussed above that are monitored by camera(s) 98. In some embodiments, controller 58 may send a snap shot one or more sensor readings (in addition to, or in lieu of, the video and/or audio-clips) in response to detecting audio and/or video events of interest.

Controller 58 of patient support apparatus 20 is adapted, in some embodiments, to communicate with one or more external sensors 122. Such external sensors 122 are adapted to detect when a patient may be undertaking an action that puts themselves, and/or others, at risk of harm. In some embodiments, external sensors 122 include one or more conventional door-ligature alarms that detect when a weight is applied to a door, such as any of the doors that may be in the patient's room (or otherwise accessible by the patient). Such conventional door-ligature alarms are 122 are configured to communicate with controller 58 via network transceiver 90 and/or by other means. Controller 58, in some embodiments, forwards this alarm information (as well as location information derived from location beacon 114) to patient support apparatus server 86. Alternatively, or additionally, such door alarms 122 may be configured to communicate with patient support apparatus server 86 by directly communicating with one or more access point 112. Patient support apparatus server 86, as will be discussed below, is configured to forward information about a door alarm 122 to one or more display devices 104.

As noted, the door-ligature alarms 122 may be conventional ligature door alarms. Examples of such conventional door-ligature alarms include those sold by Safehinge Primera of Boston, Massachusetts; those sold by Piedmont Door Solutions of Charlotte, North Carolina; and/or Door Control Services of Austin, Texas (see, e.g. U.S. Pat. No. 8,646,206 issued on Feb. 11, 2014, to Gilchrist, the complete disclosure of which is incorporated herein by reference). Still other types of ligature sensors for doors may be used and/or still other types of external sensors 122 may be used that communicate with patient support apparatus 20 and/or patient support apparatus server 86.

Patient support apparatus 20 may also include one or more caregiver presence sensors 100. In one embodiment, caregiver presence sensors 100 include one or more near field sensors that are adapted to detect near field cards, tags, or the like that are carried by caregivers. In another embodiment, caregiver presence sensors 100 are RF ID sensors that are adapted to detect RF ID cards, tags, or the like that are worn or carried by caregivers. In still another embodiment, caregiver presence sensors 100 may include one or more of the cameras 98 (visible light and/or infrared light) that have fields of view in the areas adjacent patient support apparatus 20 and are able to detect the presence of a caregiver within those fields of view. One example of a patient support apparatus 20 having such cameras built into it is found in commonly assigned U.S. Pat. No. 9,814,410 issued to Kostic et al. and entitled PERSON SUPPORT APPARATUS WITH POSITION MONITORING, the complete disclosure of which is incorporated herein by reference. In still other embodiments, one or more caregiver presence sensors 100 may be incorporated into caregiver assistance system 106 that are not positioned on patient support apparatus 20. For example, one or more cameras 98 may be positioned within the room in which patient support apparatus 20 is located and adapted to capture images of the caregivers, when present, and report that information to patient support apparatus server 86. One such suitable camera system is disclosed in commonly assigned U.S. Pat. No. 10,121,070 issued to Derenne et al. and entitled VIDEO MONITORING SYSTEM, the complete disclosure of which is incorporated herein by reference.

In some embodiments, patient support apparatus 20 may constructed to include one or more ultra-wideband (UWB) transceivers that are adapted to detect the nearby presence of caregiver's who wear UWB-equipped badges 164. In such embodiments, patient support apparatus 20 is adapted to detect when a caregiver is positioned in the same room as patient support apparatus 20 using UWB communications with the UWB-equipped badges 164. One example of such a patient support apparatus with UWB sensors that communicate with a UWB-equipped badges is disclosed in commonly assigned, U.S. patent application Ser. No. 63/356,061 filed Jun. 28, 2022, by inventors Krishna Bhimavarapu et al. and entitled BADGE AND PATIENT SUPPORT APPARATUS COMMUNICATION SYSTEM, the complete disclosure of which is incorporated herein by reference. Still other types of caregiver presence detectors 100 may be utilized, either in lieu of, or in addition to, the caregiver presence sensors 100 discussed herein.

Badges 164 may be badges of the type sold or marketed by Stryker Corporation of Kalamazoo, Michigan, under the names Vocera Badge, Vocera Smartbadge, and/or Vocera Minibadge. Other types of badges 164 may also, or alternatively, be used. Such badges 164 include the ability to transmit voice communications of healthcare workers to other badges 164 and/or other locations within a healthcare facility. Some of the badges 164 may also include text messaging abilities, alarm notifications, and other functions. As discussed above, such badges 164 may be modified to include one or more ultra-wideband transceivers that communicate with ultra-wideband transceivers onboard patient support apparatus 20 and/or built into location beacon 114. Patient support apparatus 20 and/or location beacons 114 may be configured to repetitively determine the location of any of the badges 164 that are positioned within range of its ultra-wideband transceivers and determine the location and/or orientation of such badges 164.

Patient support apparatus 20 includes a brake 64 that, when applied, prevents one or more of wheels 24 from rotating. When brake 64 is not applied, wheels 24 are free to rotate. Controller 58 communicates with brake sensor 66 (FIG. 3) and brake sensor 66 informs controller 58 whether or not brake 64 is activated or deactivated. The data from the brake sensor 66 is forwarded by controller 58 to software application 110 of patient support apparatus server 86 via network transceiver 90. Software application 110 shares this information with caregivers via one or more of the display devices 104 that are in communication with server 86, as will be discussed in greater detail below.

Patient support apparatus 20 includes two different types of actuators for turning brake 64 on and off: mechanical brake actuator 68 and electrical brake actuator 82. The mechanical brake actuator 68 may be a conventional mechanical brake actuator, such as one or more pedals that are positioned around the periphery of base 22 and that, when pressed, selectively activate and deactivate brake 64. Electrical brake actuator 82 may be a conventional electrical brake actuator, such as a button, touchscreen control, switch, etc. that, when pressed, causes the brake 64 to be selectively activated and deactivated. In some situations, it is desirable for a caregiver to be able to prevent the patient from deactivating brake 64, such as situations where there is a risk that the patient may use movement of patient support apparatus 20 across the floor to injure themselves, others, and/or cause property damage. In such situations, patient support apparatus 20 is configured to allow the caregiver to disable the mechanical brake actuator 68. This is achieved through mechanical brake disabler 84. In other words, when a caregiver, or other authorized individual, activates mechanical brake disabler 84, mechanical brake actuator 68 is no longer operative. The patient therefore cannot use the brake pedals (or other mechanical controls) to deactivate (or activate) the brake 64.

Access to the functionality of mechanical brake disabler 84 is obtained via control panel 54a. Accordingly, if control panel 54a is operating in the blocked mode, a patient will not be able to access the disabler 84. Similarly, in some embodiments, access to electrical brake actuator 82 is prevented when control panel 54a is in the blocked mode. In this manner, if the caregiver activates brake 64 (through either electrical or mechanical means), then activates brake disabler 84, and control panel 54a thereafter switches to the blocked mode of operation (either through a time window of disuse expiring or the caregiver actively putting control panel 54a in the blocked mode through block access control 50g), the patient will be prevented from deactivating brake 64 because they will not be able to access either electrical brake actuator 82 or mechanical brake disabler 84 (and because mechanical brake actuator 68 will be disabled). Patient support apparatus 20 therefore allows the caregiver to prevent unauthorized individuals, such as the patient, from changing the desired state of brake 64.

Patient support apparatus 20 communicates with the software application 110 of patient support apparatus server 86 via local area network 56 (FIG. 3). Software application 110 is adapted to assist the caregivers in performing a plurality of tasks. In general, software application 110 is adapted to assist the caregivers in ensuring that the patient support apparatuses 20 are maintained in a desirable state, to assist the caregivers in alerting them of undesired conditions associated with their patients and/or patient support apparatuses 20, and/or to assist in other ways. In some embodiments, software application 110 may additionally, or alternatively, implement one or more functions of the hostile person detection system 300, as will be discussed in greater detail below. Alternatively, the hostile person detection system 300 may be implemented separately from caregiver assistance system 106 and/or software application 110.

In order to carry out its functions, software application 110 may include, or utilize, a set of local rules (local to a particular healthcare facility, or portion of a healthcare facility), a data repository, a communication interface, and/or a web Application Programming Interface. The set of local rules may be defined prior to the installation of software application 110 within a particular healthcare facility, and/or it may be modifiable by authorized personnel after installation within the healthcare facility. Such modifications are made by way of one or more computers 134 (FIG. 4) that are in communication with local area network 56 and that act as user interfaces for software application 110. Thus, an authorized individual 136 (FIG. 4) may utilize computer 134 to communicate with software application 110 and add, delete, or modify one or more of the local rules.

The local rules may include, but are not limited to, the following: rules indicating what state patient support apparatuses 20 are to be placed in; rules defining situations detected by one or more sensors onboard patient support apparatuses 20 (e.g. sensors 66, 94, 96, 98, 100, etc.) that are indicative of undesired patient agitation, undesired risks of harm (to self, others, or property), and/or other undesired situations; rules specifying who is to be notified, and when, if an undesired situation and/or undesired state of patient support apparatuses is detected; rules specifying how such notifications are to be communicated (e.g. email, phone call, texts, etc.); rules specifying what personnel within the healthcare facility are authorized to view what data using software application 110; and/or other rules. As will be discussed in greater detail below, the rules defining situations that present undesired risks of patient harm to self, others, and/or to property, as well as any of the other rules, may be modified by authorized individuals 136 to vary based upon one or more factors. For example, these rules may be modified for different wings of the healthcare facility, different units of the healthcare facility, different patients, different patient conditions, different patient assessments, different times of day and/or different shifts, different models of patient support apparatuses, different patient treatments, different data stored in an EMR server 124, etc.

The local rules may also include additional administrative data that is stored on patient support apparatus server 86, or stored in a memory otherwise accessible to software application 110. Such administrative data includes, but is not limited to, the IP address, or other network address, of each of the servers with which software application 110 is to communicate (e.g. an EMR server 124 and/or other servers), and/or the IP addresses or other configuration data necessary for software application 110 to communicate with one or more middleware software applications that act as gateways to one or more of these servers. The administrative data also may also include the email addresses, passwords, phone numbers, caregiver badge IDs, user names, access levels, and other information about those hospital personnel who have been authorized to use software application 110. The email address and/or phone numbers are used in some embodiments of software application 110 that are configured to automatically send alerts to one or more caregiver tags and/or to one or more display devices 104.

The communication interface used by software application 110 controls the communications between software application 110 and the display devices 104 with which it is in communication (FIGS. 3-4). The communication interface may also control the communications between software application 110 and the servers with which it is in communication. All of these communications, in at least one embodiment, are carried out using conventional Internet packet routing. That is, patient support apparatuses 20 send data in packets that have an IP address corresponding to patient support apparatus server 86, and server 86 sends message packets back to patient support apparatuses 20 that include an IP address corresponding to the particular patient support apparatus(es) 20 to which the messages are intended. In some embodiments, each patient support apparatus 20 includes a static IP address that is stored on the patient support apparatus 20, while in other embodiments, the patient support apparatuses 20 consult a local Dynamic Host Configuration Protocol (DHCP) server (not shown) on local area network 56 and the DHCP server assigns a network address to the patient support apparatuses 20.

When communicating with other servers within the healthcare facility, the communication interface of software application 110 may utilize different communication protocols, such as, but not limited to, Link Layer Protocol (LLP), Hyper-Text Transfer Protocol Secure (HTTPS), and/or Simple Mail Transfer Protocol (SMTP), etc. In order to facilitate the communication between patient support apparatus server 86 and the other servers of local area network 56, the communication interface may utilize a conventional interface engine, such as, but not limited to, the Redox cloud platform that is commercially available from Redox, Inc. of Madison, Wisconsin. Alternatively, or additionally, the communication interface may utilize a conventional iGUANA interface engine (HL-7 or otherwise) available from INTERFACEWARE, Inc. of Toronto, Ontario. Such interfaces allow software application 110 to communicate with different types and/or brands of Electronic Health Record (EHR) systems, such as, but not limited to, those marketed by Cerner corporation, Epic Corporation, Allscripts, etc.

The web API that may be used in some embodiments of software application 110 provides a portal for authorized devices, software applications, and/or servers to access the data of software application 110. In some embodiments, display devices 104 communicate with software application 110 via the web API by using a web browser built into the display devices 104 that accesses one or more Uniform Resource Locators (URLs) that direct the web browser to software application 110. The web API, in some embodiments, uses JavaScript Object Notation (JSON) to communicate with the web browsers of the display devices 104. In other embodiments, the web API use Extensible Markup Language (XML) to communicate with the web browsers of the display devices 104. Still other types of communication may be used.

In some embodiments, the web API may be configured to communicate with the display devices 104 using the conventional GET, POST, DELETE, and UPDATE verbs of the Hyper-Text Transfer Protocol (HTTP). These are used for providing RESTful service (i.e. Representational State Transfers) between web API and the display devices 104. For those aspects of software application 110 that utilize two way interactive communication, conventional web socket protocols (e.g. IETF RFC 6455, or the WebSocket API in Web IDL (Interface Description Language) that is standardized by the World Wide Web Consortium (W3C)) may be used for communication between the web API and the display devices 104. Alternatively, or additionally, conventional pull and push requests may be used for this communication, as well as, but not limited to, server-sent events and/or long polling. Still other communication techniques may be used. In some embodiments, such communications are encrypted such that at least those messages containing patient data are secured against interception. Such encryption takes place, in at least one embodiment, as part of a RESTulf Web service (RWS).

In general, software application 110 performs the following functions: gathers data from patient support apparatuses 20 about their current states, their assigned patients, and/or the environment in which the patient support apparatus 20 is positioned; communicates this data to display devices 104 that are remote from patient support apparatus server 86; causes the display devices 104 to display one or more notifications regarding the current state of the patient support apparatuses 20, their patients, and/or their environments; and/or performs other functions. In some embodiments, software application 110 may be configured to perform any one or more of the functions and/or algorithms performed by the caregiver assistance system disclosed in commonly assigned PCT patent application serial number PCT/US2021/033408, filed May 20, 2021, by applicant Stryker Corporation and entitled CAREGIVER ASSISTANCE SYSTEM, the complete disclosure of which is incorporated herein by reference.

Patient support apparatus 20 is shown in FIGS. 3-4 in communication with local area network 56 of the healthcare facility. It will be understood that the precise structure and contents of the local area network 56 will vary from healthcare facility to healthcare facility. FIG. 4 illustrates in greater detail the contents of a common hospital's local area network 56, along with patient support apparatus server 86 and several display devices 104.

As shown in FIG. 4, local area network 56 may include a plurality of servers, including a conventional Admission, Discharge, and Tracking (ADT) server 140, a conventional nurse call server 142, a conventional Electronic Medical Records server 124, and a plurality of conventional wireless access points 112. Local area network 56 also includes patient support apparatus server 86 that, together with one or more patient support apparatuses 20 and one or more display devices 104, implement one embodiment of the caregiver assistance system 106 according to the present disclosure. Still further, network 56 includes a conventional Internet gateway 144 that couples local area network 56 to the Internet 146, thereby enabling the servers and/or patient support apparatuses 20 to communicate with computers outside of the healthcare facility, such as, but not limited to, a geographically remote server 148. In some embodiments, all or some of the functions of software application 110 of patient support apparatus server 86 are carried out by geographically remote server 148, while in other embodiments software application 110 of patient support apparatus server 86 is configured to implement all or some of its functions without accessing geographically remote server 148.

ADT server 140, which may be a conventional server, stores patient information, including the identity of patients and the corresponding rooms 126 and/or bays within rooms to which the patients are assigned. That is, ADT server 140 includes a patient-room assignment table 150 (FIG. 4), or functional equivalent to such a table. The patient-room assignment table 150 correlates rooms, as well as bays within multi-patient rooms, to the names of individual patients within the healthcare facility. The patient's names are entered into the ADT server 140 by one or more healthcare facility staff whenever a patient checks into the healthcare facility and the patient is assigned to a particular room within the healthcare facility. If and/or when a patient is transferred to a different room and/or discharged from the healthcare facility, the staff of the healthcare facility update ADT server 140. ADT server 140 therefore maintains an up-to-date table 150 that correlates patient names with their assigned rooms. ADT server 140 may be a conventional server marketed by Cerner Corporation of North Kansas City, Missouri; EPIC Systems of Madison, Wisconsin; Allscripts Healthcare Solutions, Inc. of Chicago, Illinois; and/or by other companies. Still other types of ADT servers 140 may, of course, be used. In some embodiments, ADT server 140 and/or a portion of its functions may be integrated into, or combined with, those of EMR server 124.

EMR server 124 (FIGS. 3-4) stores the medical records of individual patients. Such patient records identify a patient by name and the medical information associated with that patient. Such medical information may include all of the medical information generated from the patient's current stay in the healthcare facility as well as medical information from previous visits. An abbreviated EMR table 152 (FIG. 4) shows an abbreviated example of one type of medical information that may be entered into a patient's medical records: a risk assessment of the patient's risk of harming themselves, others, and/or property. Although FIG. 4 shows this data expressed as text, it will be understood that this data may be stored within a medical record in numeric format. For example, the risk of harm (to themselves, others, and/or property) data may be stored as a numeric value generated from a healthcare facility's assessment of the patient.

As noted, a typical EMR server 124 will include far more additional information in the medical records of each patient than what is shown in table 152 of FIG. 4. It will be understood that the term “EMR server,” as used herein, also includes Electronic Health Records servers, or EHR servers for short, and that the present disclosure does not distinguish between electronic medical records and electronic health records. EMR server 124 may be a conventional server marketed by Cerner Corporation of North Kansas City, Missouri; EPIC Systems of Madison, Wisconsin; Allscripts Healthcare Solutions, Inc. of Chicago, Illinois; and/or by other companies. Still other types of EMR servers 124 may, of course, be used.

Nurse call server 142 is shown in FIG. 4 to include a caregiver assignment table 154 that matches caregivers to specific rooms and/or bays within the healthcare facility. Although table 154 only shows caregivers assigned to a single room, it will be understood that each caregiver is typically assigned to multiple rooms. In some nurse call systems, caregivers are assigned to specific patients, rather than to specific rooms. Caregiver assistance system 106 is configured to work with both types of nurse call systems. Caregiver assistance system 106 is also adapted to work with healthcare facilities that utilize a separate caregiver assignment server (not shown), rather than nurse call server 142, to assign caregivers to rooms and/or patients. Nurse call server 142 may be a conventional server marketed by Rauland (now owned by Ametek, Inc. of Berwyn, Pennsylvania); by West-Com Nurse Call System, Inc. of Fairfield, California; and/or by other companies.

Regardless of whether caregiver assignment table 154 is stored within nurse call server 142 or some other server on network 56, nurse call server 142 is configured to communicate with caregivers and patients. That is, whenever a patient on a patient support apparatus 20 presses, or otherwise activates, a nurse call control, the nurse call signals pass through nurse call cable 130 to nurse call outlet 128. Nurse call outlet 128 is coupled via wire to nurse call server 142 and/or to another structure of the nurse call system that then routes the call to the appropriate nurse. The nurse is thereby able to communicate with the patient from a remote location.

Local area network 56 may include additional structures not shown in FIG. 4, such as, but not limited to, one or more conventional work flow servers and/or charting servers that monitor and/or schedule patient-related tasks for particular caregivers, and/or one or more conventional communication servers that forward communications to particular individuals within the healthcare facility, such as via one or more portable devices (smart phones, pagers, beepers, laptops, etc.). The forwarded communications may include data and/or alerts that originate from patient support apparatuses 20 as well as data and/or alerts that originate from patient support apparatus server 86.

Wireless access points 112 are configured, in at least some embodiments, to operate in accordance with any one or more of the IEEE 802.11 standards (e.g. 802.11g, 802.11n, 802.11ah, etc.). As such, patient support apparatuses 20 and display devices 104 that are equipped with Wi-Fi capabilities, and that have the proper authorization credentials (e.g. password, SSID, etc.), can access local area network 56 and the servers hosted thereon. This allows patient support apparatus 20 to send messages to, and receive messages from, software application 110 of patient support apparatus server 86. This also allows display devices 104 to send messages to, and receive messages from, software application 110 of patient support apparatus server 86. As noted previously, alternatively, or additionally, patient support apparatuses 20 may include a wired port for coupling a wired cable (e.g. a Category 5, Category 5e, etc.) between the patient support apparatus 20 and one or more routers/gateways/switches, etc. of network 56, thereby allowing patient support apparatuses 20 to communicate via wired communications with software application 110 of server 86.

In still other embodiments, one or more of the patient support apparatuses 20 are equipped with alternative wireless transceivers enabling them to communicate directly with patient support apparatus server 86 via an antenna and transceiver that is directly coupled to server 86 and that is separate from LAN 56, thereby allowing patient support apparatuses 20 to bypass LAN 56 in their communications with server 86. One example of patient support apparatuses equipped to communicate directly with a server on a healthcare facility's local area network without utilizing the LAN is disclosed in commonly assigned U.S. patent application Ser. No. 15/831,466 filed Dec. 5, 2017, by inventors Michael Hayes and entitled NETWORK COMMUNICATION FOR PATIENT SUPPORT APPARATUSES, the complete disclosure of which is incorporated herein by reference. In some embodiments, patient support apparatuses 20 include communication modules, such as the communication modules 66 disclosed in the aforementioned '466 application, and server 86 is coupled directly to a receiver, such as the enterprise receiver 90 disclosed in the aforementioned '466 application. In such embodiments, patient support apparatuses 20 are able to both send and receive messages directly to and from server 86 without utilizing access points 112 or any of the hardware of network 56 (other than server 86).

Software application 110 of patient support apparatus server 86 is configured to construct a table 156 (FIG. 4), or a functionally equivalent type of data structure, that determines in which specific rooms 126—and/or bays within the rooms of the healthcare facility—each of the patient support apparatuses 20 is currently located. Software application 110 determines these room and/or bay locations by using the known location of each location beacon 114 within the healthcare facility (which may be determined via a surveying procedure during the installation of beacons 114) and the known beacon IDs of each location beacon 114. Each location beacon 114 sends a unique beacon ID 160 (FIG. 4) to an adjacent patient support apparatus 20 when the patient support apparatus 20 is positioned within a close proximity of the patient support apparatus 20. The patient support apparatus 20, in turn, forwards this unique beacon ID 160, along with its unique patient support apparatus ID 158, to software application 110. Software application 110 then uses these two IDs, along with the known location of the locator beacons 114, to determine the location of each patient support apparatus 20.

Software application 110 also receives status conditions from each patient support apparatus 20. Such status conditions may include data from any of the various sensors onboard patient support apparatus 20, including data relating to the condition of the patient, data relating to the condition of patient support apparatus 20, and/or data relating to the environment (e.g. room) in which patient support apparatus 20 is positioned. Each patient support apparatus 20 sends these status conditions to software application 110 with its corresponding unique patient support apparatus ID 158. Software application 110 is therefore able to correlate incoming patient support apparatus status conditions with specific patient support apparatuses 20 and specific locations within the healthcare facility. In other words, software application 110 is able to construct a data structure like table 156 of FIG. 4, which includes the room location and status conditions for each of the patient support apparatuses 20 within the healthcare facility (or within a portion of the healthcare facility).

Although not shown in table 156 of FIG. 4, software application 110 may also correlate the information of table 156 to one or more additional pieces of information, such as specific caregivers, specific patients, and/or other pieces of information. For example, software application 110 may determine which caregivers are associated with each of the patient support apparatuses 20 based on the caregiver-to-room assignment data it receives from nurse call server 142 (i.e. the data of table 154). By using this caregiver-to-room assignment data, software application 110 is able to determine which caregiver(s) are assigned to each of the patient support apparatuses 20. Further, software application 110 may determine which patients are associated with each of the patient support apparatuses 20 based on the patient-to-room assignment data it receives from ADT server 140 (i.e. the data of table 150). By using this patient-to-room assignment data, software application 110 is able to determine which patient is assigned to each of the patient support apparatuses 20. Further, by knowing which patient is assigned to each patient support apparatus 20, software application 110 is able to assign medical information received from EMR server 124 to each of the patient support apparatuses 20. Such medical information may include information indicative of the risk of harm of the patient (e.g. the risk of harm to themselves, others, and/or to property) In summary, software application is supplied with sufficient data to know the current status of each patient support apparatus 20, the room in which each patient support apparatus 20 is assigned, the caregiver assigned to that room and/or patient support apparatus 20, the patient assigned to each patient support apparatus 20, and the harm risk and/or other medical data of each patient.

In some embodiments, software application 110 is configured to determine patient-to-room, patient-to-bed, patient-to-bed-bay, patient-to-caregiver, caregiver-to-room, caregiver-to-patient-support-apparatus, and/or caregiver-to-bed-bay correlations in any of the manners disclosed in commonly assigned U.S. patent application Ser. No. 62/826,097, filed Mar. 29, 2019 by inventors Thomas Durlach et al. and entitled PATIENT CARE SYSTEM, the complete disclosure of which is incorporated herein by reference. In some embodiments, software application 110 may further be modified to carry out any of the staffing errors, and other error-notification functions, disclosed in the aforementioned '097 application.

Display devices 104 (FIGS. 3-4) may come in a variety of different forms. As shown in FIG. 4, some display devices 104 are mobile display devices 104a intended to be carried by a user (e.g. caregiver) while other display devices 104 are stationary display devices 104b that generally remain in one location. Mobile display devices 104a may take on different forms, such as, but not limited to, smart phones, tablets, laptop computers, badges 164 (FIG. 4), Computers on Wheels (COWs), and others. Stationary display devices 104b may also take on different forms, such as, but not limited to, smart televisions, displays, Personal Computers (PCs), and others. For purposes of the following written description, reference to display devices 104 will refer to both display devices 104a and 104b, unless otherwise stated. Also, for purposes of the following written description, caregiver assistance system 106 will be described with reference to display devices 104 that communicate with software application 110 via a conventional web browser. It will be understood, however, that in other embodiments, some or all of the display devices 104 may be modified to execute a specialized or native software application that is downloaded to the display device 104 and that is tailored to be executed by the particular operating system of the display device (e.g. Android, IOS, Windows, etc.). The specialized software application is executed by the microcontroller(s) of the display device 104 and carries out the functions of caregiver assistance system 106 described herein.

In some embodiments, in order for a caregiver associated with a display device 104 to access caregiver assistance system 106, the caregiver utilizes the web-browsing application contained within the display device 104 to go to a particular web page, or other URL, associated with software application 110. Any conventional web-browsing software may be used for this purpose, including, but not limited to, Microsoft's Bing or Internet Explorer web browsers, Google's Chrome web browser, Apple's Safari web browser, Mozilla's Firefox web browser, etc. The particular URL accessed with the web browser may vary for different healthcare facilities and can be customized by authorized IT personnel at the healthcare facility. In some embodiments, a domain name may be associated with software application 110 that is resolved by a local DNS server to the IP address of patient support apparatus server 86 (e.g. www.caregiver-assistance-app.com). In other embodiments, display devices 104 may include their own native software applications that are programmed to interact with software application 110, thereby avoiding the usage of a web browser to access software application 110. Access to software application 110 may be achieved in other manners. As noted, the following description will focus primarily on using a conventional web browser onboard display devices 104 to access the caregiver assistance application, but it will be understood that display devices 104 may include their own software apps that are specifically tailored to interact with software application 110.

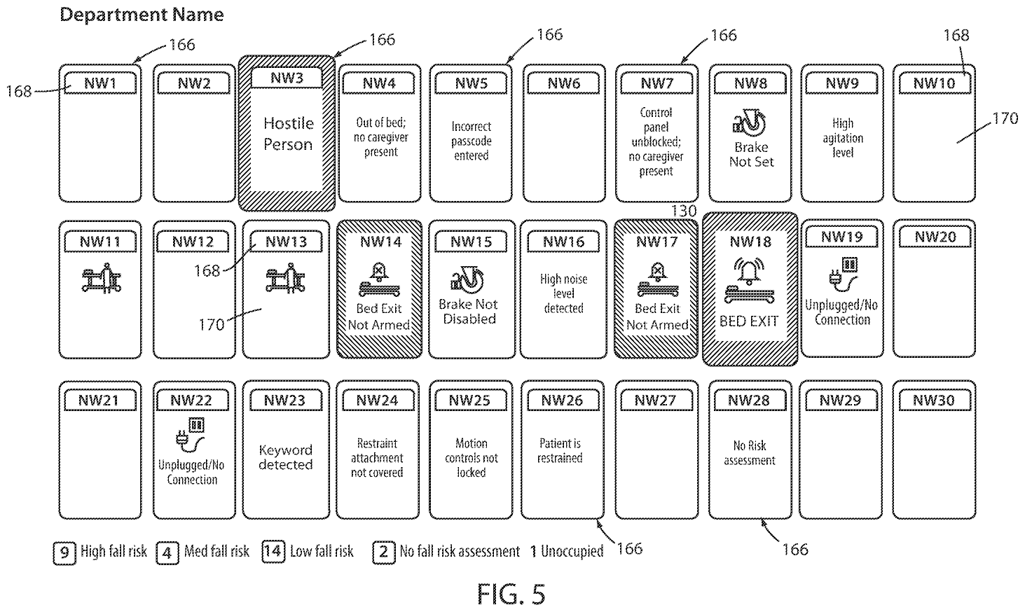

Software application 110 may be configured to require a user to enter a user name and/or password via the display device 104 before the user is able to access software application 110. After entering the appropriate information into a display device 104, the software application 110 is configured to instruct the display device 104 to display data regarding one or more patient support apparatuses 20 and/or one or more patients that are positioned within the healthcare facility. Such data may include a dashboard screen, such as the dashboard screen 162 of FIG. 5.

Dashboard screen 162 is particularly suited for being displayed on display devices 104 that have a relatively large display size, such as stationary display devices 104b (and not, for example, mobile display devices 104a that have a relatively small screen, such as smart phones or small computers). Dashboard screen 162 includes a plurality of room icons 166 (i.e. enclosures that are defined by rectangles having rounded corners). Each room icon 166 corresponds to a particular room and/or bay within an actual room of the healthcare facility in which caregiver assistance system 106 is installed. Thus, in the example shown in FIG. 5, there are thirty room icons 166. Each room icon 166 includes a header portion 168 that identifies the particular room in the healthcare facility to which the room icon 166 corresponds and a body portion 170 that, as will be discussed more below, may display information about the status of the patient and/or the patient support apparatus 20 positioned within that particular room.

As shown in FIG. 5, header portion 168 is color coded. That is, software application 110 in configured to instruct the display device 104 to display header portion 168 in different colors depending upon the harm risk of the patient assigned to that particular room. In the example dashboard screen 162 of FIG. 5, header portion 168 of room NW1 has a green background, which indicates that the patient in room NW1 has a low risk of committing harm to themselves, others, or to property. In contrast, the patient assigned to room NW5 in the example of FIG. 5 has a medium harm risk, and software application 110 is configured to instruct display device 104 to display the header portion 168 for room NW5 with a blue background. In the example of FIG. 5, software application 110 instructs display device 104 to display header portion 168 with a yellow background for those patients having a high fall risk (e.g. rooms NW7, NW8, etc.), and a gray background for those patients whose fall risk has not yet been determined (e.g. NW25, NW 28). As shown by room NW2 of screen 162, software application 110 may instruct display device 104 to omit header portion 168 in those rooms for which no patient is assigned (or to use a white background that blends in with the white background of body portion 170).

Software application 110, in at least some embodiments, determines the harm risk of a particular patient by receiving this information from EMR server 124 and/or ADT server 140. ADT server 140 and/or EMR server 124 may also contain requirements data identifying one or more protocols that the healthcare facility requires its caregivers to follow when caring for one or more patients. Such requirements data, for example, may specify what assessments are to be performed on a patient, such as an assessment of the patient's risk of harm, fall risk and/or bed sore risk. Alternatively, such requirements data may be stored elsewhere, such as, but not limited to, the local rules of software application 110. In some embodiments, the requirements data that specifies which assessments (harm, fall, skin, etc.) are to be performed for a given patient may depend upon the location of the patient within the healthcare facility. For example, some healthcare facilities may configure the local rules of software application 110 such that all patients within a particular wing, floor, or other section, receive a harm assessment, while patients in other areas of the healthcare facility receive a fall risk and/or bed sore risk assessment and/or none of these assessment. Software application 110 automatically checks these local rules when a new patient is admitted to the healthcare facility (as determined from communication with ADT server 140) and, if no assessment is recorded in EMR server 124, it may be configured to display a reminder on dashboard screen 162 and/or send an alert to one or more mobile devices associated with the patient whose assessment has not been completed.

Thus, when a new patient enters the healthcare facility, software application 110 automatically determines from server 140 and/or its local rules if a particular patient is supposed to have one or more risk assessments performed. If so, software application 110 further sends an inquiry to EMR server 124 to determine if such an assessment has been completed for that particular patient. If it has not, software application 110 displays this lack of completion on dashboard screen 162. In the example shown in FIG. 5, the patients in room NW25 and NW 28 have not yet had a harm risk assessment performed, and this information is shown by the color coding of header portion 168 (and in the body portion 170 of room NW28).

Software application 110 receives the harm risk assessments of individual patients from EMR server 124 and/or from ADT server 140, and uses that information both when determining what rules to apply to a particular patient's patient support apparatus 20, as well as when determining what information to display on dashboard screen 162. As was noted previously, software application 110 may be configured to display the background color of the header portions of 126 of each room icon 166 in a different color based on the corresponding patient's assessed risk of harm to self, others, and/or to property.

In some embodiments, software application 110 is configured to display different data on dashboard screen 162 for patients who have different harms risks. In other words, in some embodiments, software application 110 is configured to use different rules for determining what information to display on dashboard 162, depending upon what level of risk of harm a particular patient has been assessed to possess. Thus, for example, software application 110 may be configured to display any one or more of the following for patients at a high risk of harm: an unsuccessful attempt has been made to unblock control panel 54a; control panel 54a has been successfully unblocked but not caregiver's presence has been detected by sensor 100; the patient's agitation level exceeds a threshold (and the threshold may vary based on the harm risk level); noises above a threshold are detected by microphone(s) 94; patient support apparatus 20 is moving as detected by accelerometer(s) 96; a caregiver is present or not present, as detected by caregiver presence sensor 100; a key work or key phrase is detected by microphone(s) 94; an external sensors, such as door-ligature detector 122, detects a weight applied to a door in the vicinity of the patient; a restraint is being used with a patient; a restraint mount on patient support apparatus 20 is not covered; one or more motion controls of patient support apparatus 20 are not locked; mechanical brake actuator 68 has not been disabled; a patient is engaging one or more behaviors as detected by camera(s) 98; and/or other information. In some embodiments, the rules used by software application 110 may be customized by the user to vary based on the time of day, location within healthcare facility, and/or other factors.

Still further, software application 110 may utilize rules that combine any one or more of the conditions described herein. For example, software application 110 may be customized by a user to only display when control panel 54a is unblocked if caregiver presence detector 100 simultaneously does not also detect the presence of a caregiver. Similarly, as another example, software application 110 may be customized to only display an indicator on dashboard 162 of a sound above a threshold level if no caregiver is present within the room at the time of the sound. Other combinations can, of course, be made. Software application 110, in some embodiments, allows a user to use Boolean logic to define rules in terms of what conditions and/or combinations of conditions must be detected by the sensors discussed herein before a notification is sent by software application 110 to one or more display devices 104. And, as noted, such Boolean-defined rules may be contingent upon the harm risk of a particular patient, the time of day, the location of the patient support apparatus 20, and/or other factors.

Software application 110 is configured to use the defined rules to instruct the display devices 104 to display selected undesired patient support apparatus, patient, and/or environmental conditions in the body portions 170 of each room icon 166. Examples of undesired patient support apparatus conditions include, but are not limited to, any one or more of the following: an AC power cord 138 (FIG. 4) of the patient support apparatus 20 is not currently plugged into an AC outlet 132; one or more siderails 36 of a patient support apparatus 20 are not in their raised position; a nurse call cable 130 of is not plugged into an outlet 128; brake 64 is not activated; mechanical brake actuator 68 has not been disabled by disabler 84; exit detection system 88 is not armed (and/or not armed with the right sensitivity level); the litter frame 28 is not at its lowest height (or within a desired range of heights); the angle of the head section 44 (a.k.a. Fowler section) of patient support apparatus 20 is not above a threshold value; a restraint on patient support apparatus 20 has not been covered, thereby creating a structure for a ligature to be hung; one or more motion controls on patient support apparatus 20 have not been locked; a communication connection between patient support apparatus 20 and the nurse call outlet 128 has not been established; exit detection system 88 is currently detecting an exit alert condition; and/or still other undesired conditions.

Software application 110 is also configured to instruct display device 104 to display undesired patient and/or environmental conditions on dashboard screen 162 (FIG. 5) such as, but not limited to, any one or more of the following: the patient is out of patient support apparatus 20 (as detected by scale/exit detection system 88) and no caregiver is present in the room (as detected by caregiver presence sensor(s) 100 (see, e.g. room NW4); an incorrect passcode has been entered into control panel 54a of patient support apparatus 20 (see, e.g. room NW5); control panel 54a is not in a blocked state and no caregiver is present (see, e.g. room NW7); a patient currently has a high agitation level (see, e.g. room NW9); a high noise level has been detected in a room microphone(s) 94 (see, e.g. room NW16); the patient has uttered a key word and/or key phrase, as detected by microphone(s) 94 (see, e.g. room NW 23); and/or an assessment of the patient's risk of harm (to self, others, and/or to property) has not been performed for a particular patient (see, e.g. room NW28).

In some embodiments, in addition to the data displayed on dashboard screen 162 of FIG. 5, software application 110 may be configured to display any of the additional data displayed on the dashboard screen 162 (FIG. 4) of commonly assigned Indian patent application number 202211062036 filed Oct. 31, 2022, by inventors Sujay Sukumaran et al. and entitled CAREGIVER ASSISTANCE SYSTEM, the complete disclosure of which is incorporated herein by reference.

Dashboard screen 162 (FIG. 5) is typically-though not necessarily always-displayed on the display of stationary electronic devices 104b, rather than mobile displays 104a. This is because stationary display devices 104b typically have larger sized displays that mobile display devices 104a, and dashboard screen 162 includes a large amount of information that may be difficult to read on a mobile display device 104a having a relatively small screen. Accordingly, software application 110 may be configured to send display instructions to mobile display devices 104a that are different than the display instructions that it sends to stationary display device 104b. More particularly, software application 110 may be configured to instruct mobile display devices 104a to an abbreviated dashboard screen that contains room information for only the rooms to which a particular caregiver is assigned responsibility.

Although a typical mobile display device 104a may be associated with a particular caregiver, this is generally not true for stationary display devices 104b (FIG. 4). Stationary display devices 104b, which may include large screen smart televisions, may be associated with a particular unit of a healthcare facility, a particular nurse's station, wing, floor, and/or other section of the healthcare facility. For these devices, the login credentials may be tailored to the particular location and/or intended function of that particular stationary display device 104b. For example, a stationary display device 104b may be associated with an oncology unit, an east wing, nurse's station XYZ, the second floor, or rooms A through G, or something else. In such instances, software application 110 may be configured to assign a username and password to each such display device 104b that is custom tailored to that specific device. Thus, for example, if a particular display device 104b is positioned at a nurse's station within a pediatric oncology unit, the display device 104b may be assigned a username of “pediatric oncology display” and have its own specific password. Once an authorized user has logged into software application 110 via that device, caregiver assistance application displays the rooms and/or patient data corresponding to the pediatric oncology unit on that particular device 104b. The room and/or patient data may include rooms and/or patients that are assigned to multiple caregivers, thereby allowing the display device 104b to display information beyond that associated with a single caregiver.

Software application 110 may be configured to automatically determine which rooms a particular caregiver has been assigned by communicating with a server on local area network 56 that maintains room assignments for caregivers. In the example illustrated in FIG. 4, nurse call server 142 is shown to include a caregiver-room assignment table 154 that stores the room assignments for the caregivers within the healthcare facility. Caregiver-room assignment table 154 may also, or alternatively, be stored on a different server. During installation of software application 110, an authorized administrator inputs the IP address of the server containing caregiver room assignment table 154 (and/or other data necessary to gain access to caregiver-room assignment table 154). Similar data is also input for all of the other servers and tables discussed herein. After a particular user successfully logs into software application 110, software application 110 sends a message to the server having caregiver room assignment table 154. The message requests an up-to-date listing of the rooms that are assigned to the specific caregiver who has just logged in. After receiving this information, software application 110 may instruct the mobile display device 104a to only display those rooms on the display of the mobile display device 104a that have patients assigned to that particular caregiver.

The data displayed in dashboard screen 162 (FIG. 5) is updated in real time, or near real time. In most embodiments of patient support apparatuses 20, the patient support apparatuses 20 are configured to automatically (and nearly immediately) communicate their status and/or sensor outputs to patient support apparatus server 86 whenever a change occurs in their status and/or a sensor detects a change in a condition of the patient and/or the environment. Thus, for example, if microphone 94 detects an excessive noise level (i.e. above a threshold-which may be user-selectable), the patient support apparatus 20 sends a message automatically and almost immediately thereafter to patient support apparatus server 86. Software application 110 of patient support apparatus server 86 automatically, and immediately or nearly immediately, forwards this status update to all of the display devices 104 that are currently displaying status information for that particular room (or that are able to navigate to a page, such as on a mobile display device 104a, that displays that information). A caregiver, who may be remote from a particular room and/or a particular patient support apparatus 20, but nearby to a display device 104, thereby gets a real time, or near real time, update of the audio environment in which patient support apparatus 20 is located when utilizing software application 110.

In addition to communicating with display devices 104, software application 110 may be configured to also communicate with caregiver badges 164 (FIG. 4). Software application 110 may communicate any of the information shown in dashboard screen 162 and/or otherwise discussed herein to one or more caregiver badges 164. Software application 110 may also communicate aural alerts and/or other types of notifications to badges 164.

It will also be understood that software application 110 may be configured to instruct display devices 104 to display the above-described information in different manners. For example, in some embodiments, software application 110 sends the data defining the graphics shown in dashboard screen 162 to the corresponding display device 104 and instructs the display device 104 to display those graphics. However, in other examples, some or all of the graphics shown in the dashboard screen 162 may be stored locally in a software application executed by the display device 104 and software application 110 may instruct the display device 104 to display these graphics without having to forward these graphics to the display device. Still other manners of instructing the display devices 104 what to display may also, or additionally, be used.

FIG. 6 illustrates one example of a motion control screen 180 that may be displayed on display 52 of control panel 54a. Motion control screen 180 is only displayed after control panel 54a has been unblocked and a user navigations to motion control screen 180 (such as by pressing on control 50d of FIG. 2). In other words, the functions of motion control screen 180 are not accessible to a user when control panel 54a is in the blocked mode. Instead, the user must first unblock control panel 54a by entering the correct passcode, password, or other ID, and then can gain access to motion control screen 180.

Motion control screen 180 (FIG. 6) includes gatch lift and lower controls 50i and 50j, Fowler lift and lower controls 50k and 501, litter frame lift and lower controls 50m and 50n, CPR control 50h, and block access control 50g. When a user presses on Fowler lift control 50k, controller 58 sends a signal to a activate Fowler actuator (not shown) causing it to raise head section 44. When a user presses on gatch lower control 50j, controller 58 sends a signal to activate the Fowler actuator in the opposite direction, causing it to lower head section 44. When a user presses on knee or gatch raise control 50i, controller 58 sends a signal to activate a gatch actuator causing it to raise the junction of a seat section (not shown) with foot section 48. When a user presses on knee lower control 50j, controller 58 sends a signal to activate the gatch actuator in the opposite direction, causing it to lower the junction of the seat section and foot section 48. When a user presses on litter deck raise control 50m, controller 58 sends a signal to both lifts 26, causing them to raise the overall height of litter frame 28. When a user presses on litter deck lower control 50n, controller 58 sends a signal to both lifts 26, causing them to lower the overall height of litter frame 28. Pressing on CPR control 50h has been previously described.

When a user presses on block access control 50g, control panel 54a is immediately switched to the blocked mode and displays a screen, such as the passcode screen 70 of FIG. 2, that requires the user to enter proper credentials before being able to access the majority of the functions of control panel 54a. Block access control 50g therefore gives the user the option of immediately switching control panel 54a to the blocked mode instead of having to wait for a predetermined period of non-use of control panel 54a to expire before control panel 54a automatically switches to the blocked mode. In some embodiments, block access control 50g may be displayed on other screens, either in addition to, or in lieu of, motion control screen 180.

If the user wishes to lock out any of the controls on control panels 54b and/or 54c, the user can navigate to a lockout screen, such as the lockout screen 190 shown in FIG. 7. Lockout screen 190, like motion control screen 180, is only displayed after control panel 54a has been unblocked and a user navigations to lockout screen 190 (such as by pressing on control 50e of FIG. 2). In other words, the functions of lockout screen 190 are not accessible to a user when control panel 54a is in the blocked mode. Instead, the user must first unblock control panel 54a by entering the correct passcode, password, or other ID, and then can gain access to lockout screen 190.

Lockout screen 190 (FIG. 7) includes a Fowler lockout control 500, a gatch lockout control 50p, a lift lockout control 50q, a mechanical brake disabler control 50r, and a head of bed angle lockout control 50s. All of these controls 50o-s are, in some embodiments, toggle controls that, when repeatedly pressed, alternate between locking out (or disabling) and unlocking (or enabling) their respective controls. Thus, when Fowler lockout control 500 is first pressed, it locks out the motion controls for moving the Fowler section on control panels 54b and 54c. In this manner, the patient is prevented from using those controls to adjust the angular orientation of Fowler section 44 of patient support apparatus 20. Pressing Fowler lockout control 500 a second time unlocks the Fowler motion controls on control panels 54b and 54c. The other controls 50 on lockout screen 190 follow the same toggling behavior.

Gatch lockout control 50p locks out those controls on control panels 54b and 54c that control movement of the gatch (i.e. intersection between foot section 48 and a seat section 46 on patient support deck 30. Height control lockout 50q locks out those controls on control panels 54b and 54c that control lifts 26 and the height of litter frame 28. Mechanical brake disabler control 50r controls mechanical brake disabler 84, alternating between disabling and enabling mechanical brake actuator 68 in response to disabler control 50r being repeatedly pressed. Head of bed angle lockout control 50s locks out the motion controls on control panels 54b and 54c such that they are not able to lower Fowler section 44 below a predetermined angular orientation (such as, but not limited to, 30 degrees). Unlock control 50t, when pressed, changes any and all of the lockout controls 50o-s that were previously in the locked state to the unlocked state, thereby enabling the corresponding controls on control panels 54b, 54c.

Motion lockout screen 190 allows a user, such as a caregiver, to stop a patient from being able to activate any of the actuators onboard patient support apparatus 20, if desired. The user simply navigates to lockout screen 190, locks out the desired motion controls, and then either puts control panel 54a into the blocked state or allows control panel 54a to switch to the blocked state on its own. Because the patient does not know the correct passcode, password, and/or have the proper ID, the patient is not able to get past passcode screen 70. As a result, none of the motion controls that were locked out by the user will be accessible to the patient, and the patient is thereby prevented from activating any of the motors or other types of actuators onboard patient support apparatus 20.

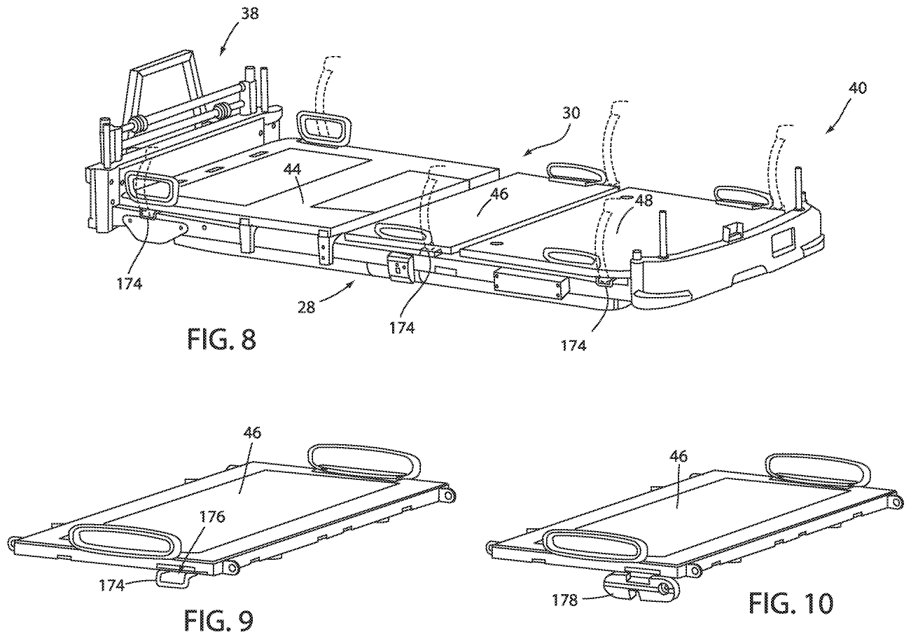

FIG. 8 illustrates one example of litter frame 28 and support deck 30. In the particular example of litter frame 28 shown in FIG. 8, litter frame 28 includes a plurality of restrain attachments 174. Each restraint attachment 174 is adapted to allow a restraint to be attached thereto. The restraints are used to restrain a patient lying on litter frame 28. Because restraint attachments 174 define an opening 176 (FIG. 9) through which one end of a restraint may be inserted, a patient intent upon self-harm could loop a sheet, shirt, or other ligature-forming material through the restraint opening 176 and use it for self-injury. As a result, in some instances, it is desirable to attach a restraint cover 178 (FIG. 10) over restraint attachment 174, thereby blocking access to opening 176 and preventing a patient from inserting a ligature through restraint opening 176.

In some embodiments, camera(s) 98 are configured to include within their field of view the restraint attachments 174 and controller 58 is configured to perform image analysis on the images captured by camera(s) 98 to determine whether or not a restraint cover 178 has been applied to each of the restraint attachments 174 of patient support apparatus 20. If controller 58's image analysis indicates that one or more restraint attachments 174 do not have restraint covers 178 attached to them, controller 58 is configured to send a message to software application 110 indicating one or more missing restraint covers 178. Software application 110, in turn, is configured to instruct one or more display devices 104 to display this information on dashboard screen 162 (e.g. room NW24 of FIG. 5). More particularly, software application 110 is configured to select the set of display devices 104 that are associated with that particular patient support apparatus 20 (e.g. the display devices 104 of caregivers assigned to that patient, the display devices 104 associated with that particular room 126, etc.), and to instruct that set of display devices to display information indicating that one or more restraint covers 178 are missing.

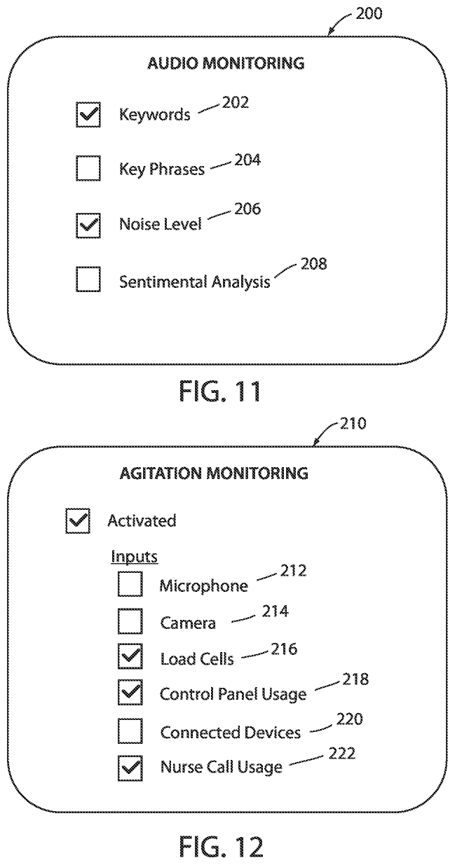

In some embodiments, controller 58 is configured to always perform image analysis of the images captured by camera(s) 98 to determine if a restraint cover 178 is attached to each restraint attachment 174. In other embodiments, controller 58 only performs this image analysis if the patient assigned to patient support apparatus 20 has a risk of harm (to self, others, or to property) that is above a threshold. In these latter embodiments, patient support apparatus server 86 may send an inquiry to EMR server 124 requesting the harm risk assessment for each patient and, if the risk of harm is above a threshold, inform the patient support apparatuses 20 that are associated with patient's whose harm risk is above a threshold. Controller 58 is configured to respond to this information by analyzing the images captured by camera(s) 98 to see if restraint covers 178 are applied to each restraint attachment 174. This process may be repeated for a variety of other conditions that controller 58 is configured to monitor. In other words, controller 58 may be configured to only monitor one or more of the conditions associated with a patient's risk of harm if it receives a message from patient support apparatus server 86 informing it that the patient assigned to that patient support apparatus 20 has an elevated risk of harm (e.g. above the threshold). Alternatively, in some embodiments, controller 58 may monitor the one or more conditions associated with a patient's risk of harm in all instances and/or in response to the caregiver activating a control onboard patient support apparatus 20 that instructs controller 58 to monitor one or more of these conditions. Several of such controls are discussed below with respect to FIG. 11.

In some embodiments, the restraint covers 178 may take on the form of the restraint covers disclosed in commonly assigned U.S. patent application Ser. No. 17/945,264 filed Sep. 15, 2022, by inventors Michael W. Graves et al. and entitled COVER SYSTEMS FOR BLOCKING APERTURES OF PATIENT SUPPORT APPARATUSES, the complete disclosure of which is incorporated herein by reference. Still other types of restraint covers may also or alternatively be used.

FIG. 11 illustrates an audio monitoring selection screen 200 that may be displayed on display 52 of control panel 54a of some embodiments of patient support apparatus 20. Audio monitoring selection screen 200 allows a caregiver to select what audio conditions will be monitored by patient support apparatus 20. Audio monitoring selection screen 200 includes a plurality of different conditions: key word monitoring 202, key phrase monitoring 204, and noise level monitoring 206, and sentiment monitoring 208. Tapping on any one or more of these conditions causes controller 58 to toggle between activating and deactivating audio monitoring for these conditions. Thus, in the example shown in FIG. 11, controller 58 has been instructed to monitor the outputs of microphone(s) 94 for the utterance of one or more keywords and for noise levels that are above a threshold. If controller 58 detects either or both conditions, it sends a message to patient support apparatus server 86 and patient support apparatus server 86 instructs the corresponding display devices 104 associated with that patient support apparatus 20 to display information about the monitored condition on dashboard 162 (see, e.g. rooms NW16 and NW23 in FIG. 5).

It will be understood that audio monitoring screen 200 may includes a lesser or greater number of conditions than the conditions 202, 204, 206, and 208 shown in FIG. 11. It will also be understood that control panel 54a may be configured to allow the user of patient support apparatus 20 to customize one or more aspects of each of the conditions that are monitored by controller 58. Thus, for example, if the user presses and holds key word condition 202 for a threshold amount of time, controller 58 may bring up a customization screen (not shown) that allows the user to type in which specific key words it will look to detect when monitoring the outputs from microphone(s) 94. The customization screen may also allow the user to specify other aspects of the monitoring, such as, for example, how many times one or more key words need to be detected in a given time period before controller 58 reports it to patient support apparatus server 86, the language of the key words that are being monitored, and/or other aspects of the key word monitoring process.

Similarly, if the user presses and holds key phrase condition 204 for a threshold amount of time (or otherwise navigates to a key phrase customization screen), controller 58 may bring up a customization screen (not shown) that allows the user to type in which specific key phrases it will look to detect when monitoring the outputs from microphone(s) 94. The customization screen may also allow the user to specify other aspects of the monitoring, such as, for example, how many times the one or more key phrases need to be detected in a given time period before controller 58 reports it to patient support apparatus server 86, the language of the key phrases that are being monitored, and/or other aspects of the key word monitoring process.

With respect to noise level condition 206, if the user presses and holds this condition 206 for a threshold amount of time (or otherwise navigates to a noise level customization screen), controller 58 may bring up a customization screen (not shown) that allows the user to specify that threshold level above which noises detected by microphone(s) 94 will be reported to patient support apparatus server 86. The customization screen may also allow the user to specify the frequencies and/or spectral content of the noises that must exceed the threshold, how long the noise must exceed the threshold and/or how many noises above the threshold need to be detected in a given time period before controller 58 reports it to patient support apparatus server 86, other characteristics of the noises, and/or other aspects of the key word monitoring process.

With respect to sentiment condition 208, which will be discussed in greater detail below, if a user presses and holds this condition 208 for a threshold amount of time (or otherwise navigates to a sentiment analysis customization screen), controller 58 may bring up a customization screen (not shown) that allows the user to specify what level of negative sentiment is required for initiating a hostile person alert, whether specific emotions are being monitored, a particular algorithm for detecting sentiment analysis, whether the sentiment analysis is based on textual analysis, audio analysis, and/or both.

As was noted, in some embodiments, controller 58 may be configured to automatically select conditions 202, 204, 206, and/or 208, as well as a set of customized aspects of these conditions, based on a message received from patient support apparatus server 86 indicating that the patient assigned to that particular patient support apparatus 20 has a harm risk above a particular threshold. This particular threshold may be customized by the healthcare facility using, for example, computer 134 to access patient support apparatus server 86. An authorized individual 136 may also use computer 134 to access patient support apparatus server 86 to determine what conditions 202, 204, 206, 208, and/or other conditions are to be monitored for patients with a harm risk above the threshold, as well as the customized aspects of those conditions. Stated alternatively, in some embodiments, patient support apparatus server 86 may be configured to display a screen similar to, if not the same as, audio monitoring screen 200 on computer 134, thereby allowing authorized administrators to control what aspects patient support apparatuses 20 will monitor for high harm risk (and/or medium and low harm risk) patients.

FIG. 12 illustrates an example of an agitation level monitoring selection screen 210 that may be displayed on display 52 of control panel 54a of some embodiments of patient support apparatus 20. Agitation level monitoring selection screen 210 allows a caregiver to select whether patient support apparatus 20 will monitor the agitation level of the patient or not. Additionally, screen 210 allows the user to select what components of patient support apparatus 20 will be used to assess the agitation level of the patient. As shown in FIG. 12, if the user activates the agitation level monitoring (as indicated by the checkmark in the box next to “activated”), the user can select any one or more six components 212, 214, 216, 218, 220, and/or 222 to use as inputs for the agitation level monitoring. These six components include a microphone component 212, a camera component 214, a load cell components 216, a control panel usage component 218, a connected device component 220, and a nurse call usage component 222.

Microphone component 212, when selected, instructs controller 58 to analyze the outputs from the microphone(s) 94 when determining the agitation level of the patient. Camera component 214, when selected, instructs controller 58 to analyze the outputs from the camera(s) 98 when determining the agitation level of the patient. Load cell component 216, when selected, instructs controller 58 to analyze the outputs from the load cells 108 when determining the agitation level of the patient. Control panel usage component 218, when selected, instructs controller 58 to analyze the usage of any one or more of the control panels 54a, b, and/or c when determining the agitation level of the patient. Connected device component 220, when selected, instructs controller 58 to analyze the outputs from one or more external sensors 122 when determining the agitation level of the patient. And nurse call usage component 222, when selected, instructs controller 58 to analyze the patient's usage of the nurse call button (not shown) when determining the agitation level of the patient. One or more nurse call buttons are typically present on the patient control panels 54c and such buttons, when pressed, allow the user to talk with a remotely positioned nurse via the nurse call outlet 128.

Each component 212-222 (FIG. 12) is selectable by the user to be a factor in the computation of an agitation level score. In some embodiments, controller 58 calculates this agitation level score, while in other embodiments, controller 58 forwards the underlying data to patient support apparatus server 86 and patient support apparatus server 86 calculates the agitation level score. As with conditions 202-206, controller 58 may be configured to automatically select a set of components 212-222, as well as a set of customized aspects of these components, based on a message received from patient support apparatus server 86 indicating that the patient assigned to that particular patient support apparatus 20 has a harm risk above a particular threshold. An authorized individual 136 may also use computer 134 to access patient support apparatus server 86 to define what components 212-222 are to be used as factors in computing the patient's agitation level score, the weighting of those factors, the formula for the agitation level score, the threshold for the score (above which causes controller 58 to send a message to patient support apparatus server 86), and other customized aspects of those components. Stated alternatively, in some embodiments, patient support apparatus server 86 may be configured to display a screen similar to, if not the same as, agitation level monitoring selection screen 21 on computer 134, thereby allowing authorized administrators to control what factors will be used for monitoring the agitation level of patients who have a high harm risk (and/or a medium or low harm risk).

As with the conditions 202, 204, 206, and 208 of audio monitoring selection screen 200, the user may customize any one or more of the agitation level components 212-222 (FIG. 12) by pressing and holding on the corresponding component 212-222. For example, if the user presses and holds microphone component 212 for a threshold amount of time, controller 58 may bring up a customization screen (not shown) that allows the user to input information defining how the outputs of microphone(s) 94 will be used for determining the patient's agitation level. The customization screen may allow, for example, the user to assign a weighting level for different levels of noise, for different key words and/or different key phrases, different times of the day, and/or for other aspects of sounds detected by microphone(s) 94.

If the user presses and holds camera component 214 (FIG. 12) for a threshold amount of time, controller 58 may bring up a customization screen (not shown) that allows the user to input information defining how the outputs of camera(s) 98 will be used for determining the patient's agitation level. The customization screen may allow, for example, the user to define what events the images captured by camera(s) 98 will be analyzed for by controller 58. For example, the user may specify whether or not the images captured by camera(s) 98 will be analyzed for any one or more of the following: rapid movement of the patient; unwanted movement of items in the room (e.g. furniture or other heavy objects); throwing objects; the patient's position is in or out of patient support apparatus 20 (and/or how frequently this changes and/or the number of times the patient enters and/or exits patient support apparatus 20); movement of patient support apparatus 20 across the floor; the position and/or orientation of the patient (e.g. if patient is upside-down, interacting with patient support apparatus 20 or other items, entrapped in patient support apparatus, etc.); the presence of ligature items and/or interaction by the patient with ligature items; thermal signatures that may indicate the presence of urine, fecal matter, blood, the sharpening of objects, etc.; and/or other events. The customization may also allow the user to specify additional factors that will be considered by controller 58 (or patient support apparatus server 86) when calculating the patient agitation level score, such as the time of day, a weighting level to be assigned to any of the events detected by camera(s) 98, and/or for other aspects of the images detected by camera(s) 98.

If the user presses and holds load cell component 216 (FIG. 12) for a threshold amount of time, controller 58 may bring up a customization screen (not shown) that allows the user to input information defining how the outputs of load cells 216 will be used for determining the patient's agitation level. The customization screen may allow, for example, the user to define what analyses will be performed on the outputs of the load cells 108 by controller 58. For example, the user may specify whether or not the outputs of load cells 108 will be analyzed for any one or more of the following: rapid movement of the patient while on patient support apparatus 20, such as indicated by the transfer of weight to/from patient support apparatus 20, rapid movement of the center of gravity of the patient, increase/decreases in the net weight detected by load cells 108, etc.; frequency of the patient getting into and out of patient support apparatus 20; patient movement indicating an intent to exit patient support apparatus 20; decreases in the net weight indicative of items being removed from patient support apparatus 20; negative or decreased net weight indicative of patient support apparatus 20 being flipped over or onto its side; load detected around the perimeter of patient support apparatus 20 that may be due to a patient applying a ligature to the patient support apparatus; the position of the patient on patient support apparatus 20; impacts of patient support apparatus 20 with objects; vibrations from impacts between objects in the environment of patient support apparatus 20; movement of patient support apparatus 20 while a caregiver is not present; the orientation of patient support apparatus 20; angular changes to patient support apparatus 20 while no actuators onboard patient support apparatus 20 are being driven; one or more vital signs of the patient; and/or other conditions. In those embodiments of patient support apparatus 20 that use load cells 108 to monitor one or more vital signs of the patient, such monitoring may be implemented in accordance with the principles disclosed in commonly assigned U.S. Pat. No. 7,699,784 issued Apr. 20, 2010, to David Wan Fong et al. and entitled SYSTEM FOR DETECTING AND MONITORING VITAL SIGNS, the complete disclosure of which is incorporated herein by reference. The customization of the analysis of the outputs of load cells 108 may also allow the user to specify additional factors that will be considered by controller 58 (or patient support apparatus server 86) when calculating the patient agitation level score, such as the time of day, a weighting level to be assigned to any of the events detected by load cells 108, and/or for other aspects of the events detected by load cells 108.

If the user presses and holds control panel usage component 218 (FIG. 12) for a threshold amount of time, controller 58 may bring up a customization screen (not shown) that allows the user to input information defining how the usage of the control panels 54a-c will be used for determining the patient's agitation level. The customization screen may allow, for example, the user to define how the frequency at which the patient uses the controls on any of the control panels 54a-c is converted into an agitation level factor. For example, repeated pressing of the same control on a control panel 54a-c may be defined to lead to a higher agitation level factor than pressing multiple different controls over the same time period. Alternatively, or additionally, the more often a control is pressed within a given time period may lead to a higher agitation level factor. The customization of the analysis of the control panel usage may also allow the user to specify additional factors that will be considered by controller 58 (or patient support apparatus server 86) when calculating the patient agitation level score, such as the time of day at which the control panels 54a-c are being used, a weighting level to be assigned to the usage of the control panels 54a-c, and/or for other aspects of the usage of control panels 54a-c.

If the user presses and holds the connected devices component 220 (FIG. 12) for a threshold amount of time, controller 58 may bring up a customization screen (not shown) that allows the user to input information defining how one or more external sensors 122 will be used for determining the patient's agitation level. The customization screen may allow, for example, the user to select different types of external sensors 122 that will be used for computing the agitation level score; to define how those external sensors 122 will be used for calculating an agitation level score, and/or to define other aspects of the external sensor(s) and their usage in computing an agitation level score. The customization of the analysis of the external sensors 122 may also allow the user to specify additional factors that will be considered by controller 58 (or patient support apparatus server 86) when calculating the patient agitation level score, such as the time of day at which the external sensor(s) 122 detect one or more events, a weighting level to be assigned to the events detected by the external sensor(s) 122, and/or for other aspects of the external sensor(s) 122.

If the user presses and holds the nurse call usage component 222 (FIG. 12) for a threshold amount of time, controller 58 may bring up a customization screen (not shown) that allows the user to input information defining how the usage of the nurse call controls on patient support apparatus 20 will be used for determining the patient's agitation level. The customization screen may allow, for example, the user to define how the frequency at which the patient uses the nurse call controls is converted into an agitation level factor. For example, the more often a nurse call control is pressed within a given time period may lead to a higher agitation level factor. The customization of the analysis of the nurse call control usage may also allow the user to specify additional factors that will be considered by controller 58 (or patient support apparatus server 86) when calculating the patient agitation level score, such as the time of day at which the nurse call controls are being used, a weighting level to be assigned to the usage of the nurse call controls, and/or for other aspects of the usage of the nurse call controls.

In some embodiments, either in addition to, or in lieu of, the calculation of an agitation level score, controller 58 and/or patient support apparatus server 86 may be configured to monitor and record all of the agitation components 212-222 and to keep track of them over a set period of time in order to bring visibility to how a patient's behavior is trending. A summary of this information over the set period of time may be displayed on control panel 54a and/or on a device coupled to patient support apparatus server 86. In some embodiments, controller 58 and/or patient support apparatus server 86 may be configured to automatically monitor this trend and to issue an alert on dashboard screen 186 if a trend is detected indicating the patient is increasingly becoming agitated. In some embodiments, agitation level monitoring screen 210 may include an additional control that allows the user to selectively turn on and off such trend monitoring, as well as to customize aspects of the trend monitoring (e.g. how much of an increase in agitation levels will trigger a notification to server 86, what components are being monitored or the trend calculations, etc.).

Software application 110 is configured, in some embodiments, to allow an authorized user, such as user 136 (FIG. 4) to customize what information is displayed on dashboard 162 and/or what information is sent to one or more mobile display devices 104a. Thus, for example, if the authorized user does not want to have dashboard 162 display a notification in response to the entry of an incorrect passcode on a patient support apparatus 20, the authorized user can configure software application 110 to omit this type of notification. The same is true for all of the other notifications discussed herein.

In addition to customizing what information is displayed on dashboard screen 162, software application 110 is also customizable by an authorized user with respect to notifications that are sent to mobile display devices 104a. That is, software application 110 not only is configured to be able to display selected notifications on dashboard 162, but it is also configured to be able to send notifications to the mobile devices 104a of specific caregivers. Thus, for example, if an authorized user wants not only dashboard 162 to display a notification that a noise level in room NW16 was detected that exceeds a threshold, but to also send a text, email, or other message to the mobile display device(s) 104a of the caregiver responsible for the patient in room NW16, the authorized user can customize software application 110 to do so. In other words, software application 110 not only allows authorized users to customize what information is displayed on dashboard 162, but also what information generates a specific communication to one or more specific mobile display devices 104a.

In some embodiments, controller 58 may be configured to automatically switch to a panic mode of operation if data from one or more of its sensors (e.g. camera(s) 98, microphone(s) 94, etc.) indicates that an emergency situation is happening. In this panic mode, the outputs from the microphone(s) and/or camera(s) 98 may be sent to patient support apparatus server 86 for display on dashboard screen 162. That is, instead of a textual or visual notification of the kind shown in FIG. 5, dashboard screen 162 may display the actual video detected by camera(s) 98 and/or output the actual audio signals detected by microphone(s) 94, thereby letting a caregiver viewing screen 162 to hear and/or see what is currently happening in the room in which the emergency is detected.

In some embodiments, system 106 may be configured to automatically monitor one or more of the conditions discussed above and to automatically determine if a patient's risk of harm (to self, others, and/or to property) should be adjusted from what was previously recorded in EMR server 124 and/or to determine an initial patient's risk of harm (where no previous assessment was performed). In such situations, if a patient's agitation level remains above a threshold for a defined amount of time, this may prompt patient support apparatus server 86 to display an indicator on dashboard 162 and/or to send a notification to one or more caregiver's mobile display devices 104a indicating that the patient's risk level may need to be changed, assessed, and/or re-assessed.

In some embodiments, system 106 may also be configured to use the images captured by camera(s) 98 to record how long a patient is restrained and/or unrestrained. System 106 may then present information on dashboard 162 and/or on other pages viewable through display devices 104 that indicate a patient's current restraint status, time of restraints, frequency of restraints, history of restraints, and/or other information regarding the restraining of the patient. Such information may also be automatically forwarded by patient support apparatus server 86 to EMR server 124.

In those embodiments where patient support apparatus 20 and/or software application 110 are configured to determine an agitation level of the patient, the agitation level may be computed in a variety of different manner. In one embodiment, after the user defines what component(s) are to be used in determining the patient's agitation level (and, in some cases, assigns a weighting to each component), controller 58 and/or software application 110 calculate an agitation level by, in response to detecting the presence of one or more components, multiplying the weighting for those component(s) by a value assigned to those component(s), summing the values of all of these products, and then comparing the result to one or more numeric thresholds. In some embodiments, different numeric thresholds may be used for different qualitative measurements of the agitation level e.g. low, medium, high.

In some embodiments, controller 58 and/or software application 110 may use a leaky bucket algorithm, or the like, wherein the agitation level is decremented over time. In such embodiments, if a patient engages in one or two relatively low-scoring behaviors that are components of the agitation level, the score generated by those behaviors will subside over time. Only if the patient engages in a high-scoring behavior, or a plurality of low-scoring behaviors over a relatively small time period, will the agitation level exceed a threshold that might lead to a separate notification to a caregiver of the patient's agitation level. As one example, if the patient presses once on the CPR button 50h while no caregiver is present, this will lead to the generation of a particular score for his or her agitation level (if the CPR button 50h is set as a component for the agitation level). That particular score will slowly dissipate over time such that, if a relatively long time (e.g. an hour or other amount) passes until the patient next presses on CPR button 50h, this will not yield an agitation level, on its own, that results in notification of the caregiver (depending upon how the user has configured software application 110). However, if the patient presses repeatedly on CPR button 50h within a relatively short period of time (e.g. seconds or minutes), each button pressing will result in an addition to the patient's agitation score, and will likely lead to a notification being sent to one or more display devices 104.

In some embodiments, repetition of certain behavior within a given time period may lead to higher scores being assigned to the repeated actions when computing the agitation level. For example, the first time the patient presses the CPR button 50h may result in a score of X being added to the patient's agitation level. If the patient presses the CPR button 50h a second time within a given time period of the first pressing, this may result in a score greater than X being added to the patient's agitation level. If the patient presses the CPR button 50h a third time within a time period, a still greater score may be added to the patient's agitation level. It will be understood that this higher weighting of repeated actions may be applied for other actions besides the patient's pressing of CPR button 50h.

It will also be understood that, when computing the patient's agitation level, different scoring may be used for the same action, depending upon whether or not a caregiver is present. In some instances, if a caregiver is present, certain actions may not affect the patient's agitation level at all, while, if those same actions are undertaken in the absence of a caregiver being present, may result in increases in the patient's agitation level.

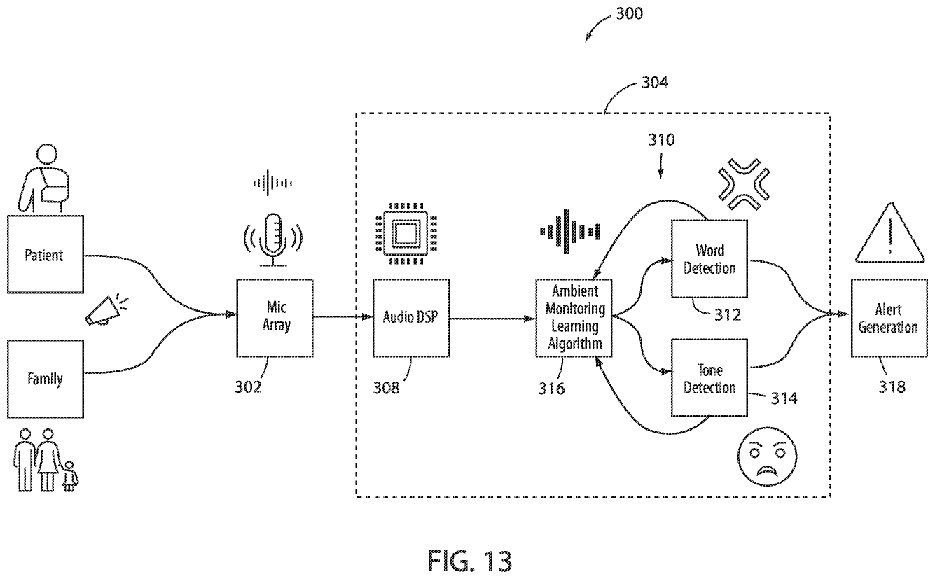

FIG. 13 illustrates a block diagram of one example of a hostile person detection system 300 according to one aspect of the present disclosure. Hostile person detection system 300 may be a subsystem of caregiver assistance system 106 (i.e. integrated therein, either wholly or partially) or it may be standalone, separate system that does not implement any of the functions of caregiver assistance system 106. Hostile person detection system 300 is designed to automatically detect the presence of one or more individuals who may be about to, or have, become hostile. In response to such detection, system 300 is designed to automatically send a notification to appropriate personnel at the healthcare facility indicating that presence of one or more hostile persons, including the location of the detected hostile person. In this manner, security or other appropriate personnel can timely respond in order to deescalate the situation and/or reduce any harm and/or damage that the hostile person might otherwise inflict.

System 300 includes a microphone array 302 and one or more computing devices 304 in communication with the microphone array 302. Microphone array 302 may comprise a single microphone or multiple microphones. Microphone array 302 may include one or more of microphones 94 from patient support apparatuses 20, one or more microphones 244 from one or more location beacons 114, one or more microphones 260 from one or more badges 164, and/or one or more standalone microphones 306 (FIG. 3). Computing device 304, in some embodiments, corresponds to patient support apparatus server 86. In other embodiments, computing device 304 may be implemented on a server located remotely from the healthcare facility but in communication with network 56. In still other embodiments, computing device 304 may be implemented, either wholly or partially, in one or more of controllers 58 of patient support apparatuses 20, and/or one or more of controllers of location beacons 114 and/or badges 164. In still other embodiments, microphone array 302 may be distributed across two or more of these aforementioned structures.

Microphone array 302 feeds audio signals to computing device 304. Microphone array 302, whether a single microphone or multiple microphones, is adapted to detect sounds in an area of interest of a healthcare facility. Generally, one microphone array 302 will be installed in each patient room 126 of a healthcare facility. However, microphone arrays 302 may be installed in other areas of the healthcare facility beyond patient rooms 126, including, but not limited to, waiting areas, hallways, and/or other areas of the healthcare facility. In general, a microphone array 302 is installed in any location where a hostile person may be present and a healthcare facility would like to detect their hostility quickly and automatically.

Each microphone arrays 302 feeds the audio signals it detects to computing device 304. Each microphone array 302 is also configured so that computing device 304 is able to determine which microphone array 302 is sending which audio signals to computing device 304. In some embodiments, as will be discussed in more detail, each microphone array 302 includes one or more transmitters that, in addition to sending audio signals of the sounds the microphone detects, also sends an ID that corresponds to that particular microphone (or array 302) to computing device 304. In this manner, computing device 304 is able to determine which audio streams correspond to which microphones and/or which microphone arrays 302.

Computing device 304 includes an audio digital signal processor 308 and a sentiment analyzer 310. Digital signal processor 308 may be a conventional audio digital signal processor adapted to mathematically manipulate the audio signals received from microphone array 203 in any manner that assists in the processing of the audio signals by sentiment analyzer 310. In the example shown in FIG. 13, sentiment analyzer includes a machine learning algorithm 316 that carries out, among other things, a word detection function 312 and a tone detection function 314. The word detection function 312 analyzes the audio signals detected by microphone array 302 and deciphers any words spoken by a person whose voice was captured by microphone array 302. The tone detection function 314 analyzes the audio signals detected by microphone array 302 and deciphers the tone of the person's voice. In some embodiments of sentiment analyzer 310, the outputs from the words and tone detection functions 312 and 314 are fed back into the machine learning algorithm 316.

Sentiment analyzer 310 is configured to perform an audio sentiment analysis on the sounds of a person's voice detected by microphone array 302. The audio sentiment analysis may utilize any of a variety of different known techniques, including any one or more of the following: automatic speech recognition using lexicon, acoustic, and/or language models to identify an anger level; raw audio waveform analysis that analyzes the raw audio output of the speaker's voice using deep neural networks to identify an anger level; and/or cross-modal bidirectional encoder representations from transformers (CM-BERT) that dynamically adjusts the weight of words through comparisons with different modalities and that allows an anger level to be identified.

In some embodiments, sentiment analyzer 310 may use any of the following three types of emotional/sentiment analysis: lexicon based, machine learning based, or deep learning based. Lexicon based emotional detection searches for keywords associated with psychological states and may use one or more established emotional lexicons, such as, but not limited to the WordNet-Affect, the National Research Council (NRC) word-emotion lexicon, and/or other lexicons. Machine learning based emotional detection utilizes one or more machine learning models that may use Naive Bayes classifiers, support vector machines, decision tree algorithms, and/or other features. Deep learning based emotional detection utilizes multiple layers of artificial neurons for detecting human emotions.

In some embodiments, sentiment analyzer 310 may utilize any one or more of the emotion identification techniques described in the article “A Review on Sentiment Analysis and Emotion Detection from Text,” written by Nandwani P and Verma R and published in Soc Netw Anal Min. 2021; 11(1):81. doi: 10.1007/s13278-021-00776-6. Epub 2021 Aug. 28. PMID: 34484462; PMCID: PMC8402961; the complete disclosure of which is incorporated herein by reference. Sentiment analyzer 310 may use still other emotional identification techniques, including not only textual emotional analysis but also audio emotional analysis.

Sentiment analyzer 310 may be specifically designed to detect the emotion or anger and to output a numerical value indicating the relative level of anger detected in the audio signals analyzed by computing device 304. Computing device 304 is configured to then compare the numeric anger level to a threshold anger level and, if it exceeds the threshold anger level, to issue a notification or alert. This notification is illustrated in FIG. 13 at step 318. In other embodiments, sentiment analyzer 310 may be configured to output a qualitative analysis of a speaker's anger and to compare it to a qualitative anger threshold. If the qualitative threshold is exceeded, computing device 304 is configured to issue the notification or alert.

In some embodiments, sentiment analyzer 310 may be configured to only use word detection function 312, while in other embodiments, sentiment analyzer 310 may be configured to use both word detection function 312 and tone detection 314 when determining a speaker's anger level. Still further, sentiment analyzer 310 may be configured to analyze the volume and/or pitch of a speaker's voice when determining the emotional state of a person.

Computing device 304 is customizable so that authorized individuals of the healthcare facility can select which individual(s) should receive the hostile person notification at step 318. The notification of step 318 may include one or more text messages, emails, voice messages, phone calls, and/or other kinds of message sent to one or more specific computers, email addresses, phones, display devices 104a,b, and/or badges 164. Authorized individuals can customize computing device 304 by defining rules of who should receive a notification (e.g. which specific caregivers, security personnel, etc.) and how those individual(s) should be notified (text, email, phone, etc.) so that computing device 304 carries out the notifications in the manner desired by the managers of the healthcare facility. Computing device 304 may automatically determine which caregiver(s) and/or other personnel to notify based on these customized rules, as well as based upon the location of the hostile person (i.e. which room or area the hostile person is located in). Computing device 304 may send the room number (if the hostile person is located in a room) with the warning message sent at step 318 so that the recipient or viewer of the warning will know the location of the hostile person.

In some embodiments, computing device 304 is in communication with one or more display devices 104a, b and is adapted to issue an alert at step 318 by sending data to the display device(s) 104 for display thereon. One example of this is shown in FIG. 5. As can be seen therein, dashboard screen 162 includes a hostile person alert for room NW3. Thus, in this example, computing device 304 is in communication with the display device 104 on which dashboard screen 162 is displayed, and computing device 304 has detected from the audio signals coming from the microphone array 302 in room NW3 that a hostile person is present in room NW3. In some embodiments, dashboard screen 162 may display room NW3 with an enlarged size and/or in a specific color (e.g. red) to draw the viewer's attention to the fact that a hostile person has been detected. In some embodiments, a message may be sent to one or more badges 164 and/or smart phones as part of step 318 that cause the badge 164 and/or smart phone to vibrate, thereby providing a silent notification to the intended recipients.

In some embodiments, computing device 304 is configured to utilize one or more patient-to-room, patient-to-bed, patient-to-bed-bay, patient-to-caregiver, caregiver-to-room, caregiver-to-patient-support-apparatus, and/or caregiver-to-bed-bay correlations when determining who to send the notification of step 318 to. In such embodiments, computing device 304 may carry out such correlations in any of the manners disclosed in either or both of the following: commonly assigned U.S. patent application Ser. No. 62/826,097, filed Mar. 29, 2019 by inventors Thomas Durlach et al. and entitled PATIENT CARE SYSTEM, and commonly assigned Indian patent application serial number 202211062036 filed Oct. 31, 2022, in the Indian Patent Office by inventors Thomas Durlach et al. and entitled CAREGIVER ASSISTANCE SYSTEM, the complete disclosures of both of which are incorporated herein by reference.

In order to determine the location of one or more of the microphones in microphone array 302, computing device 304 utilizes location information from location beacon 114 and/or other location sources. For example, when location beacons 114 are initially installed in a healthcare facility, their locations are surveyed and stored in a memory accessible to computing device 304. When a microphone 344 of a location beacon 114 sends its audio signals to computing device 304 (such as via network transceiver 246), the location beacon 114 includes a unique ID with the audio signals that uniquely identifies that particular location beacon 114. Computing device 304 therefore knows the location of the audio signals detected by that particular microphone 244.

In a similar manner, patient support apparatus 20 is able to detect its location relative to a nearby location beacon utilizing location transceiver 92. Consequently, any audio signals generated from one or more microphones 94 positioned on patient support apparatus 20 will have their location known. That is, controller 58 is adapted to include a unique ID with the audio signals it sends to computing device 304 that identifies the current location of patient support apparatus 20 based upon patient support apparatus 20's communication with a nearby location beacon 114. In some embodiments, controller 58 may also send a unique ID that uniquely identifies the particular patient support apparatus 20 that is sending the audio signals to computing device 304.

The location of badges 164 may be determined in a similar manner to the location of patient support apparatuses 20. That is, each badge 164 may include a location transceiver 262 that is adapted to communicate with a nearby location beacon 114. From this communication, the location of badge 164 is determined by a controller 264 of the badge 164 (and/or by the location beacon 114 itself). If a microphone 260 of badge 164 is sending audio signals to computing device 304 for analysis, controller 264 may include a unique ID with the audio signals that identifies the current location of badge 164. Badge 164 sends the audio signals to computing device 304 using an onboard network transceiver 266 that is adapted to communicate with network 56. Network transceiver 266 may be of the same type of transceiver as transceivers 246 and/or 90.

Hostile person detection system 300 (FIG. 13) may also include one or more standalone microphones 306 (FIG. 3) that are not incorporated into another device (e.g. patient support apparatus 20, badge 164, location beacon 114). Such standalone microphones 306 may include a transmitter 320 that is adapted to transmit audio signals from microphone 306 to computing device 304. Transmitter 320 may be the same as any of network transceivers 246, 90, and/or 266. In some embodiments, transmitter 320 may be capable of only transmitting audio signals, and not adapted to receive communications. In other embodiments, transmitter 320 may be adapted to both transmit and receive signals, and thus act as a transceiver.

Standalone microphones 306 may be installed at fixed locations within a healthcare facility, and such locations are recorded and made available to computing device 304. In some versions, transmitter 320 may include a unique ID in the audio signals it sends from microphone 306 to computing device 304, thereby informing computing device 304 of the specific microphone 306 the audio signals are coming from, as well as informing computing device 304 of the location of that specific microphone. In this manner, computing device 304 is able to determine the location of the all of the standalone microphones 306 within a healthcare facility.

In some embodiments of system 300, computing device 304 is configured to triangulate a location of the hostile person using the amplitude of the voice signals detected by the multiple microphones of array 302. In some such embodiments, array 302 consists exclusively of microphones that are positioned at known and fixed locations and orientations (e.g. standalone microphones 306 and/or microphones 244 of location beacons 114). In such cases, computing device 304 uses the known position and orientation of these microphones to triangulate a position of the voice of the hostile person. In other embodiments of array 302, array 302 may include one or more microphones whose position and/or orientation is variable (e.g. microphone(s) 94 of patient support apparatus 20 and/or microphone(s) 260 of badges 164). In these latter cases, the position and orientation of patient support apparatus 20, including its microphones 94, may be determined through the use of multiple location transceivers 92 that communicate with location unit 114. In one such example, patient support apparatus 20 includes multiple UWB location transceivers 92 that are positioned at known locations on patient support apparatus 20 and these all range with a UWB transceiver (location transceiver 238) of location beacon 114. From this multiple ranging, the position and orientation of patient support apparatus 20 can be determined. Further details of exemplary manners by which patient support apparatus 20 can use UWB transceivers to determine its location and orientation within a room are disclosed in commonly assigned PCT patent application serial number PCT/US2022/043585 filed Sep. 16, 2022, by inventors Kirby Neihouser et al. and entitled SYSTEM FOR LOCATING PATIENT SUPPORT APPARATUSES, and U.S. patent application Ser. No. 63/597,412, filed Nov. 9, 2023, by inventors Michael Graves et al. and entitled PATIENT SUPPORT APPARATUS WITH ENVIRONMENTAL INTERACTION, the complete disclosures of both of which are incorporated herein by reference. It will be understood that the use of the term “triangulate” or its variants herein is intended to include trilateration and any similar techniques.

After determining the position and orientation of patient support apparatus 20, controller 58 of patient support apparatus 20 can determine the position and orientation of each microphone 94 positioned thereon by consulting memory 60, which contains data defining the position and orientation of each microphone 94 on patient support apparatus 20. Thus, once the position and orientation of patient support apparatus 20 is determined with respect to an area of the healthcare facility, controller 58 can use the microphone position and orientation data stored in memory 60 to determine the position and orientation of each microphone 94 positioned on patient support apparatus 20. Controller 58 may forward this position and orientation information to computing device 304 which uses it when configured to triangulate a position of the hostile person.

In some embodiments, badges 164 may include two or more location transceivers 262, and in such embodiments, the location transceivers 262 may be UWB transceivers. In such embodiments, the position and orientation of a badge 164, as well as its onboard microphone(s) 260, may be determined by patient support apparatus 20 using its location transceivers 92. That is, controller 58 may use multiple UWB location transceivers 92 to range with the multiple UWB location transceivers 262 onboard badge 164 to determine the position and orientation of the badge. This location and orientation information is then sent to computing device 304 for use in its triangulation calculations. Controller 58 may send this position and orientation information directly to computing device 304, or it may send it to badge 164 and badge 164 may then forward it to computing device 304 using network transceiver 266.

In some embodiments of system 300 that are adapted to triangulate a position of a hostile person, it may be helpful to calibrate the microphone array 302 by first moving a known sound source (i.e. a sound source with known amplitude and directionality) through known positions in the environment of microphone array 302. The sound from the sound source is detected by the microphone array 302 and computing device 304 then triangulates a position of the sound source. The known position of the sound source is compared to the triangulated position and any differences are used to calibrate the triangulation method used by computing device 304.

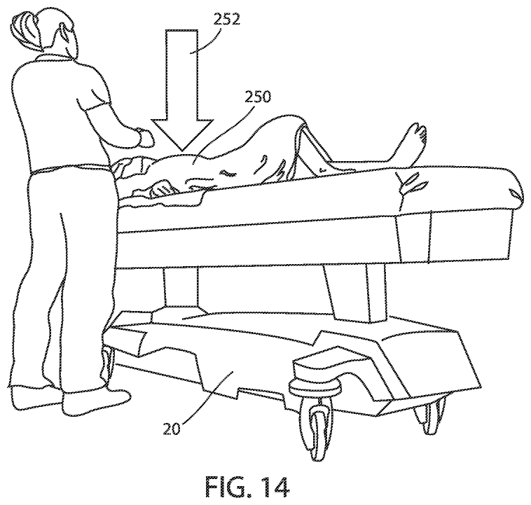

In some embodiments of system 300, one or more cameras may be included to capture images of a person whose voice is being analyzed for potential hostility. In such embodiments, system 300 may include one or more cameras 98 positioned on patient support apparatus 20, one or more cameras 242 positioned onboard location beacons 114, and/or one or more cameras 268 positioned onboard badges 164. Such cameras form a camera array similar to the microphone array 302. Computing device 304 may use the known position and orientation of the cameras to blend and/or stitch the images together. Alternatively or additionally, computing device 304 may combine the location of the hostile person as determined from the triangulation of the audio signals with the known position and orientation (and field of view) of the camera images to determine where in the images the hostile person is located. In such embodiments, computing device 304 may be configured to add, or superimpose, a marker on the image at a location corresponding to the location of the hostile person. One example of this is shown in FIG. 14, which shows patient 250 lying on a patient support apparatus 20 and surrounding by three healthcare workers. An indicator 252 in the form of a thick arrow has been added by computing device 304 in a manner that points to the patient 250, indicating that a level of hostility above a threshold has been detected in that particular patient. Computing device 304 may display the image, or images, from one or more of the cameras, including the indicator 252, on any of display devices 104a, b. The images may be still images and/or video images.

In some embodiments, one or more cameras may be utilized by system 300 to provide an automated STAMP analysis. STAMP is an acronym for Staring, Anxiety, Mumbling, and Pacing. Through sentiment analyzer 310 and an analysis of the images from the cameras for staring and pacing, computing device 304 can automate a STAMP analysis and issue a notification to caregivers at step 318 that indicates that a person may be hostile or in danger of taking a hostile action.

In some embodiments, hostile person detection system 300 may be turned on and off for a given room 126 (or area surrounding a patient support apparatus 20) through screen 200 (FIG. 11). When sentiment monitoring 208 is checked, this activates hostile person detection system 300 for that room 126 and/or area in which patient support apparatus 20 is located. When sentiment monitoring 208 is unchecked, this deactivates hostile person detection system 300 for that room 126 and/or area. This allows hostile person detection system 300 to be turned on in certain areas of the healthcare facility and turned off in other areas. Of course, the entire system may be turned on and off through any authorized electronic device in communication with computing device 304 (e.g. computer 134). In some embodiments, hostile person detection system 300 may replace the condition monitoring options 202, 204, and 206 (FIG. 11), while in other embodiments, system 300 may be included with any one or more of options 202, 204, and/or 206. Hostile person detection system 300 may be a part of caregiver assistance system 106, or it may be an entirely separate system.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1. A patient support apparatus comprising:
a support surface adapted to support a patient;
a control panel adapted to allow access to a plurality of functions of the patient support apparatus for a window of time after a correct passcode is entered into the control panel, and to disallow access to the plurality of functions after the window of time expires;
a network transceiver adapted to communicate with a healthcare facility computer network; and
a controller adapted to send a first message to the healthcare facility computer network when the correct passcode is entered into the control panel and to send a second message to the healthcare facility computer network when the window of time expires.

2. The patient support apparatus of claim 1 wherein the controller is further adapted to send a third message to the healthcare facility computer network when an incorrect passcode is entered into the control panel, the third message indicating that an incorrect passcode was entered into the control panel.

3. The patient support apparatus of claim 1 wherein the control panel includes a touchscreen and the control panel is adapted to display a passcode entry screen on the touchscreen.

4. The patient support apparatus of claim 1 further comprising a microphone, wherein the controller is adapted to send a third message to the healthcare facility computer network when a sound level detected by the microphone exceeds a threshold.

5. The patient support apparatus of claim 1 further comprising a microphone, wherein the controller is further adapted to perform speech recognition on sounds detected by the microphone and to send a third message to the healthcare facility computer network if the controller recognizes speech of a keyword.

6. The patient support apparatus of claim 5 wherein the controller is further adapted to send the third message to the healthcare facility computer network if the controller recognizes speech of any one or more of a plurality of keywords.

7. The patient support apparatus of claim 5 wherein the controller is further adapted to allow a user to change the keyword.

8. The patient support apparatus of claim 1 wherein the window of time is defined as a set time period during which the control panel is not used.

9. The patient support apparatus of claim 1 further comprising a plurality of additional control panels and the window of time is defined as a set time period during which neither the control panel nor any of the plurality of additional control panels are used.

10. The patient support apparatus of claim 1 wherein the control panel includes a block control and, in response to a user activating the block control, the window of time expires and the control panel is adapted to block access to the plurality of functions.

11-42. (canceled)

43. A patient support apparatus comprising:
a support surface adapted to support a patient;
a network transceiver adapted to communicate with a healthcare facility computer network; and
a controller adapted to send a first message to the healthcare facility computer network, wherein the first message indicates a first condition and the first condition includes at least one of the following: an incorrect passcode was entered into a control panel on the patient support apparatus; a correct passcode was entered into the control panel; a window of time since any control panel on the patient support apparatus was last used has expired; a microphone on the patient support apparatus detected a sound level exceeding a threshold; the microphone on the patient support apparatus detected a keyword; a block control was activated on the patient support apparatus wherein the block control prevents access to a plurality of functions until the correct passcode is entered; an agitation level of the patient onboard the patient support apparatus; a patient activation of a nurse call control onboard the patient support apparatus; a restraint status indicative of whether the patient is currently restrained or not; a restraint cover status indicative of whether a restraint attachment on the patient support apparatus is currently covered or not; or movement of the patient support apparatus as detected by an accelerometer.

44. The patient support apparatus of claim 43 wherein the controller is adapted to send a second message to the healthcare facility computer network, wherein the second message indicates a second condition and the second condition includes at least one other of the following: an incorrect passcode was entered into the control panel; the correct passcode was entered into the control panel; the window of time expired; the microphone on the patient support apparatus detected a sound level exceeding the threshold; the microphone on the patient support apparatus detected the keyword; the block control was activated on the patient support apparatus; the agitation level of the patient onboard the patient support apparatus; the patient activation of the nurse call control onboard the patient support apparatus; the restraint status indicative of whether the patient is currently restrained or not; the restraint cover status indicative of whether the restraint attachment on the patient support apparatus is currently covered or not; or movement of the patient support apparatus as detected by the accelerometer.

45. The patient support apparatus of claim 43 wherein the control panel is adapted to allow access to a plurality of functions of the patient support apparatus for the window of time after the correct passcode is entered into the control panel, and to disallow access to the plurality of functions after the window of time expires, and wherein the control panel includes a touchscreen and the control panel is adapted to display a passcode entry screen on the touchscreen.

46. The patient support apparatus of claim 43 wherein the first message indicates a sound level exceeding the threshold was detected by the microphone.

47. The patient support apparatus of claim 43 wherein the first message indicates the keyword was detected by the microphone.

48. The patient support apparatus of claim 47 wherein the controller is further adapted to allow a user to change the keyword.

49. The patient support apparatus of claim 45 wherein the first message indicates the window of time expired.

50. The patient support apparatus of claim 49 wherein the window of time is defined as a set time period during which the control panel is not used.

51. The patient support apparatus of claim 49 further comprising a plurality of additional control panels and the window of time is defined as a set time period during which neither the control panel nor any of the plurality of additional control panels are used.

52. The patient support apparatus of claim 49 wherein in response to a user activating the block control, the window of time expires and the control panel is adapted to block access to the plurality of functions.

53-114. (canceled)
